(19) United States
(12) Patent Application Publication
(10) Pub. No.: US 2023/0414626 A1
(43) Pub. Date: Dec. 28, 2023

(54) INDUCTION OF FERROPTOSIS FOR CANCER THERAPY

(71) Applicant: Memorial Sloan Kettering Cancer Center, New York, NY (US)

(72) Inventors: Xuejun Jiang, New York, NY (US); Craig B. Thompson, New York, NY (US); Jiajun Zhu, New York, NY (US); Junmei Yi, New York, NY (US)

(21) Appl. No.: 18/249,248

(22) PCT Filed: Oct. 16, 2021
(86) PCT No.: PCT/US2021/055326

§ 371 Date: Apr. 15, 2023

Related U.S. Application Data
(60) Provisional application No. 63/093,151, filed on Oct. 16, 2020.

(51) Int. Cl. A61K 31/5377; A61K 31/4375; A61K 31/437; A61K 31/436; C12N 15/113; A61P 35/00; A61K 31/501; A61K 31/4439

(52) U.S. Cl. CPC A61K 31/5377; A61K 31/4375; A61K 31/437; A61K 31/436; C12N 15/113; A61P 35/00; A61K 31/501; A61K 31/4439; C12N 2310/531

(57) ABSTRACT

The present invention provides methods for the treatment of cancers by administration of inhibitors of the PI3K-AKT-mTORCl pathway (or components downstream of this pathway such as SREBP and SCD1) and agents that induce ferroptosis to subjects in need thereof. The present invention also provides various related compositions and related methods.

### CROSS-REFERENCE TO RELATED APPLICATIONS

This application claims the benefit of priority of U.S. Provisional Patent Application No. 63/093,151 filed on Oct. 16, 2020, the content of which is hereby incorporated by reference in its entirety.

### STATEMENT REGARDING FEDERALLY SPONSORED RESEARCH

This invention was made with government support under grant numbers CA201318, CA204232 and CA008748 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Oct. 15, 2021, is named MSKCC_051_WO1_SL.txt and is 4,615 bytes in size.

### INCORPORATION BY REFERENCE

For the purpose of only those jurisdictions that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention.

### BACKGROUND

Accumulation of phospholipid peroxides, byproducts of cellular metabolism, can lead to an iron-dependent form of cell death referred to as ferroptosis (1, 2). Although the physiological function of ferroptosis is still obscure, its involvement in various pathological conditions, including ischemic organ injury, neurodegeneration, and cancer has recently been demonstrated (1, 6-8). Particularly, mounting evidence indicates that ferroptosis may play a role in tumor suppression (9-12) and that activation of ferroptosis may contribute to responses to some cancer treatments, such as immune checkpoint blockade (13) and radiotherapy (14-16). Importantly, cancers of mesenchymal property or harboring E cadherin-NF2-Hippo pathway mutations have been shown to be highly susceptible to cell death by ferroptosis, due to the alterations of in redox/iron homeostasis and metabolic processes related to ferroptosis (17-19). However, for most cancers little is known about whether ferroptosis plays a role in disease. Similarly, little is known about whether particular tumorigenic mutations or genetic backgrounds may play a role in the sensitivity of particular cancers to ferroptosis.

### SUMMARY OF THE INVENTION

The present invention is based, in part, on a series of important discoveries that are described in more detail in the Examples section of this patent specification. For example, it has now been discovered that activation of the PI3K-AKT-mTORC1 signaling pathway enables cancer cells to become resistant to ferroptosis by upregulating downstream SREBP1-mediated lipogenesis. Furthermore, and importantly, it has now also been discovered that administration inhibitors of the PI3K-AKT-mTORC1 pathway (or components downstream of this pathway such as SREBP and SCD1) together with agents that induce ferroptosis to mammalian subjects leads to significant regression of tumors that have an activating mutation in the PI3K-PTEN-AKT-mTOR pathway—thus providing a new therapeutic approach for a large and important group of cancers. Building on these discoveries, and other discoveries presented herein, the present invention provides a variety of new and improved methods for the treatment of various cancers.

Accordingly, in some embodiments the present invention provides methods for treating a tumor in a mammalian subject in need thereof, the methods comprising administering to the subject an effective amount of both (a) an inhibitor of the PI3K/Akt/MTOR signaling axis and (b) an inducer of ferroptosis, thereby treating the tumor in the subject.

In other embodiments the present invention provides therapeutic compositions comprising: (a) an inhibitor of the PI3K/Akt/MTOR signaling axis, (b) an inducer of ferroptosis and (c) a therapeutically acceptable carrier, for use in treatment of a tumor in a subject in need thereof.

In yet other embodiments the present invention provides the combination of (a) an inhibitor of the PI3K/Akt/MTOR signaling axis and (b) an inducer of ferroptosis, for use in treatment of a tumor in a subject in need thereof.

In some embodiments the subject has a tumor with an activating mutation in the PI3K-PTEN-AKT-mTOR pathway. In some embodiments the subject has a tumor with a PTEN deletion. In some embodiments the subject has a breast tumor. In some embodiments the subject has a breast tumor with an activating mutation in the PI3K-PTEN-AKT-mTOR pathway. In some embodiments the subject has a prostate tumor. In some such embodiments the subject has a prostate tumor with a PTEN deletion.

In some embodiments the inhibitor of the PI3K/Akt/MTOR signaling axis is selected from the group consisting of a PI3K inhibitor, an Akt inhibitor, an MTOR inhibitor, an MTORC1 inhibitor, an SREBP inhibitor and SCD1 inhibitor.

In some embodiments the inhibitor of the PI3K/Akt/MTOR signaling axis is a PI3K inhibitor. In some embodiments the PI3K inhibitor is selected from the group consisting of GDC-0941, SAR245409, SAR245408, BYL-719, GDC-0980, wortmannin, Ly294002, demethoxyviridin, perifosine, delalisib, idelaisib, PX-866, IPI-145, BAY 80-6946, BEZ235, RP6530, TGR 1202, RP5264, SF1126, INK1117, BKM120, Palomid 529, GSK1059615, ZSTK474, PWT33597, IC87114, TG100-115, CAL263, RP6503, PI-103, GNE-477, CUDC-907 and AEZS-136.

In some embodiments the inhibitor of the PI3K/Akt/MTOR signaling axis is an Akt inhibitor. In some embodiments the Akt inhibitor is selected from the group consisting of MK-2206, MK-2206, perifosine, GSK690693, ipatasertib (GDC-0068), AZD5365, afuresertib (GSK2110183), At13148, PF-04691502, AT7867, triciribine, CCT128930, A-674563, PHT0427, miltefosine, honokiol, and TIC10.

In some embodiments the inhibitor of the PI3K/Akt/MTOR signaling axis is an MTOR inhibitor. In some embodiments the MTOR inhibitor is selected from the group consisting of SAR245409, GDC-0980, CCI-779, KU-0063794, rapamycin, epigallocatechin gallate (EGCG), caffeine, curcumin, resveratrol, sirolimus, temsirolimus, everolimus, and ridaforolimus.

In some embodiments the inhibitor of the PI3K/Akt/MTOR signaling axis is an MTORC1 inhibitor. In some embodiments the MTORC1 inhibitor is selected from the group consisting of Temsirolimus (CCI-779), Torin and a short hairpin RNA (shRNA) inhibitor of RAPTOR.

In some embodiments the inhibitor of the PI3K/Akt/MTORC1 signaling axis is a an SREBP inhibitor. In some embodiments the SREBP inhibitor is Fatostatin A.

In some embodiments the inhibitor of the PI3K/Akt/MTORC1 signaling axis is an SCD1 inhibitor. In some embodiments the SCD1 inhibitor is CAY10566.

In some embodiments the inducer of ferroptosis is selected from the group consisting of RSL3, erastin, imidazole ketone erastin (IKE), sulfasalazine, sorafenib, altretamine, artesunate, ML-162 and ML-210.

These and other aspects of the present invention are described further in the below Detailed Description, Drawings, Examples and Claims sections of this patent disclosure. Furthermore, one of skill in the art will recognize that the various embodiments of the present invention described throughout this patent disclosure can be combined in various different ways, and that such combinations are within the scope of the present invention.

### BRIEF DESCRIPTION OF THE DRAWINGS

FIG. 1 A-D. Oncogenic activation of the PI3K-AKT-mTOR signaling pathway confers resistance to ferroptosis. (A) Genetic background of the analyzed cancer cell lines and their sensitivity to RSL3. (B) Cells were seeded in 96-well plate, 2×104 cells per well and incubated overnight. Cell death was induced by 24-h treatment of RSL3 with indicated concentrations. Cell death were measured by Sytox Green staining, as detailed in Methods. (C) Indicated protein components in the PI3K-AKT pathway were detected by western blot in indicated cell types. (D) Cells were treated with or without PI3K inhibitor GDC-0941 (2 μM), AKT inhibitor MK-2206 (2 μM), RSL3 (1 μM), or ferroptosis inhibitor Ferrostatin-1 (Fer-1, 1 μM) as indicated for 12 h (BT474) or 24 h (MDA-MB-453). Cell death was measured. Indicators for P values (same in all other figures): ****, P≤0.0001; *** P≤0.001; **, P≤0.01; *, P≤0.05; ns, P>0.05

FIG. 2 A-E. mTORC1, instead of mTORC2, suppresses ferroptosis. (A) MDA-MB-453 and BT474 were treated with CCI-779 (0.5 μM), RSL3 (1 μM for MDA-MB-453 cells and 0.5 μM for BT474 cells), and Fer-1 (1 μM) as indicated. (B) Cells were seeded in 6-well plate, 4×105 cells per well and incubated overnight. MDA-MB-453 and BT474 cells were treated as indicated. CCI-779, 0.5 μM; RSL3, 1 μM for MDA-MB-453 cells and 0.5 μM for BT474 cells; Fer-1, 1 μM. Cells were stained with 5 μM C1I-BODIPY followed by flow cytometry after 8 h-treatment. (C) 3D spheroids were treated as indicated. CCI-779, 0.5 μM; RSL3, 0.5 μM; Fer-1, 1 μM. Top panel, dead cells were stained by SYTOX Green (scale bar, 100 μm). Bottom panel, cell viability was assayed by measuring cellular ATP levels. (D) Cells expressing shRNAs targeting RPTOR or RICTOR were treated as indicated. CCI-779, 0.5 μM; RSL3, 0.5 μM; Fer-1, 1 μM. (E) Lipid peroxidation of samples as in panel D were measured.

FIG. 3 A-E. NRF2 is not the major mediator of the ferroptosis-suppressing activity of mTORC1. (A) BT474 cells were treated as indicated for 8 h. RSL3, 0.5 μM; Torin, 1 μM. Western blot was performed to measure p-T389 S6, total S6K and NRF2. (B) NRF2 was depleted by CRISPR/Cas9 technology in HT1080 cells. NRF2 level was measured by western blot. (C) Control or NRF2-depleted cells were treated as indicated. Erastin, 0.5 μM; RSL3, 25 nM. (D) HepG2 cells with or without NRF2 depletion were treated with cysteine starvation with or without CCI-779 (0.5 μM) as indicated. Sytox Green were added after 48 h for cell death staining (scale bar, 100 m). (E) PC-3 cells with or without NRF2 depletion were treated with cysteine starvation with or without CCI-779 (0.5 μM) as indicated. Sytox Green were added after 48 h for cell death staining (scale bar, 100 m).

FIG. 4 A-F. mTORC1 activation suppresses ferroptosis by upregulating SREBP1. (A) BT474 and MDA-MB-453 cells were treated as indicated. RSL3, 0.5 μM; CCI-779, 0.5 μM. Cell lysates were collected after 8 h and 24 h of treatment for BT474 cells and MDA-MB-453 cells, respectively, for western blot detecting p-T389 S6, total S6K, unprocessed SREBP1 (SREBP1(p)) and processed, mature SREBP1 (SREBP1(m)). (B) Cells were treated as indicated. RSL3, 0.5 μM for BT474 cells and 1 μM for MDA-MB-453 cells; Fer-1, 1 μM. (C) Cells were treated as in panel B. Lipid peroxidation was measured. (D) 3D spheroids derived from BT474 cells harboring control or SREBF1 sgRNA were treated as indicated. CCI-779, 0.5 μM; RSL3, 0.5 μM; Fer-1, 1 μM. Top, cell death stained by Sytox Green (scale bar, 100 μm). Bottom, cell viability. (E) SREBP1m was overexpressed in BT474 cells and determined by western blot. Cells were treated as indicated. RSL3, 0.5 μM; CCI-779, 0.5 μM. Cell death was measured (bottom panel). (F) 3D spheroids derived from BT474 cells were treated as indicated. RSL3, 0.5 μM; CCI-779, 0.5 μM; Fer-1, 1 μM. Top, cell death stained by Sytox Green (scale bar, 100 m). Bottom, cell viability.

FIG. 5 A-H. SREBP1 protects cells from ferroptosis through SCD1 activity. (A) The expression of SREBP1, and its targets SCD1, FASN, and ACACA, in control and SREBF1-sgRNA cells were detected by western blot. (B) The mRNA level of SREPF1 and its targets gene SCD were measured by RT-PCR. (C) Cells were pretreated with or without 5 μM CAY10566 overnight, and then subjected to the indicated treatments. RSL3, 0.5 μM for BT474 cells and 1 μM for MDA-MB-453 cells; CAY10566, 5 μM; Fer-1, 1 μM. (D) Cells expressing control or SCD-sgRNA were treated as indicated. RSL3, 1 μM for MDA-MB-453 cells and 0.5 μM for BT474 cells; Fer-1, 1 μM. (E) 3D spheroids derived from BT474 cells harboring control or SCD-sgRNA were treated as indicated. RSL3, 0.5 μM; CCI-779, 0.5 μM; Fer-1, 1 μM. Left, cell death staining (scale bar, 100 m). Right, cell viability. (F) SCD1 was overexpressed in BT474 cells and determined by western blot. Cells were treated as indicated. RSL3, 0.5 μM; CCI-779, 0.5 μM. (G) 3D spheroids derived from BT474 cells with control or SCD1 overexpression were treated as indicated. RSL3, 0.5 μM; CCI-779, 0.5 μM; Fer-1, 1 μM. Left, cell death staining (scale bar, 100 m). Right, cell viability. (H) Cells were treated as indicated. RSL3, 0.5 μM for BT474 and 1 μM for MDA-MB-453; CCI-779, 0.5 μM; Oleic acid (OA), 0.5 mM; stearic acid (SA), 0.5 mM.

FIG. 6 A-F. Combination of mTORC1 inhibition with ferroptosis induction leads to tumor regression in vivo. (A) CRISPR/Cas9-mediated, Dox-induced GPX4 knockout (GPX4-iKO) in BT474 cells, monitored by western blot. (B) Images of resected tumors from mice xenografted with GPX4-iKO BT474 cells. Groups of mice were treated with CCI-779 and/or Dox as indicated (n=6 per group). See Methods for detail. (C) Representative haematoxylin and eosin (H&E) and immunostaining images of GPX4, Ki67, PTGS2 and pS235/236 S6, all counterstained with haematoxylin (blue), are shown from sections of xenografted tumors. Scale bar, 50 μm. (D) Growth curves of BT474 tumors of each group. Data are plotted as mean±s.d., n=6, on the linear scale for actual tumor size (upper panel) or the log 2 scale for the fold change of tumors (bottom panel). (E) Growth curves of PC-3 tumors of each group (n=6), on the linear scale for actual tumor size (upper panel) or the log 2 scale for the fold change of tumor size (bottom panel). (F) Model depicting that oncogenic activation of PI3K-AKT-mTORC1 signaling suppresses ferroptosis via SREBP1/SCD1-mediated lipogenesis.

FIG. 7 A-J. PI3K-AKT-mTOR signaling regulates ferroptosis sensitivity. (A) Cells were treated as indicated. GDC-0941, 2 μM; MK-2206, 2 μM; RSL3, 1 μM; Fer-1, 1 μM. Lipid peroxidation was measured. (B) Cells were treated with indicated conditions. Torin, 1 μM; RSL3, 1 μM for MDA-MB-453 cells and 0.5 μM for BT474 cells; Fer-1, 1 μM. Cell death was measured. (C) Cells were treated with indicated conditions. RSL3, 10 μM for MCF cells and PC-3 cells, 5 μM for T47D cells, 1 μM for HepG2 cells; Torin, 1 μM; CCI-779, 0.5 μM. (D) Cells were treated as indicated. CCI-779, 0.5 μM; Torin, 1 μM; Fer-1, 1 μM. Cell death was staining by propidium iodide (PI) (red) or Sytox Green (green) (scale bar, 100 μm). (E-F) 3D spheroids for MDA-MB-453 cells and MCF7 cells were treated as indicated. CCI-779, 0.5 μM; RSL3, 1 μM for MDA-MB-453 cells and 5 μM for MCF7 cells; Fer-1, 1 μM. Top panels, cell death staining (scale bar, 100 m). Bottom panels, cell viability. (G) MDA-MB-453 cells were treated as indicated for 24 h. RSL3, 1 μM; CCI-779, 0.5 μM; GDC-0941, 2 μM; MK-2206, 2 μM; Dabrafenib, 2 μM; SCH772984, 2 μM; Fer-1, 1 μM. (H) Western blot was performed to detect RPTOR and RICTOR knockdown efficiency. (I) HT1080 cells and MDA-MB-231 cells (both with wild-type PI3K-AKT-mTOR pathway) were treated as indicated. RSL3, 0.1 μM for HT1080 and 0.25 μM for MDA-MB-231; Torin, 1 μM; CCI-779, 0.5 μM; Fer-1, 1 μM. Cell death was measured. (J) Two lines of PI3K-AKT-mTOR pathway wild-type cells (HT1080 and MDA-MB-231) and two lines of cells harboring activating mutation of the pathway (BT474 and MDA-MB-453) were treated as indicated. RSL3, 0.25 μM; Fer-1, 1 μM. Western blot was performed to detect the level of pT389 S6K.

FIG. 8 A-D. NRF2 is not the main player mediating the ferroptosis-suppressing activity of mTORC1. (A) NRF2 was depleted by CRISPR/Cas9 technology in HepG2 cells. NRF2 level was measured by western blot. (B) NRF2 was depleted by CRISPR/Cas9 technology in PC-3 cells. NRF2 level was measured by western blot. (C) NRF2 was depleted by CRISPR/Cas9 technology in MCF7 cells. (Left) NRF2 level was measured by western blot. (Right) MCF7 cells with or without NRF2 depletion were treated as indicated. RSL3, 5 μM; CCI-779, 0.5 μM; Fer-1, 1 μM. Cell death was measured. (D) Keap1 was depleted by CRISPR/Cas9 technology in BT474 cells. (Left) NRF2 and Keap1 levels were measured by western blot. (Right) BT474 cells with or without Keap1 depletion were treated as indicated. RSL3, 0.5 μM; Torin, 1 μM; Fer-1, 1 μM.

FIG. 9 A-F. SREBP1 protects cells from ferroptosis. (A) MCF7 cells were treated as indicated. RSL3, 5 μM; CCI-779, 0.5 μM. Cell lysates were collected 24 h after treatment for Western blot detecting p-T389 S6, total S6K, SREBP1(P) and SREBP1(m). (B) Cells were pretreated with 5 μM Fatostatin A overnight and treated as indicated. RSL3, 0.5 μM for BT474 cells, 1 μM for MDA-MB-453 and 5 μM for MCF7 cells; Fer-1, 1 μM. (C) Efficiency of SREBF1 Knockout in BT474, MDA-MB-453, and MCF7 cells was monitored by western blot. (D) MCF7 cells were treated as indicated. RSL3, 5 μM; Fer-1, 1 μM. (E) Cells were treated as indicated. RSL3, 1 μM for MDA-MB-453 cells and 0.5 μM for BT474 cells; Fer-1, 1 μM; Torin, 1 μM; GDC-0941, 2 μM; MK-2206, 2 μM; CCI-779, 0.5 μM. (F) SREBP1m was overexpressed in MCF7, MDA-MB-453 and A549 cells and determined by western blot. Cells were treated as indicated. RSL3, 5 μM for MCF7 cells, 0.5 μM for MDA-MB-453 cells and 0.25 μM for A549 cells; CCI-779, 0.5 μM.

FIG. 10 A-B. SREBP1 knockout downregulates SCD1. (A) Indicated lines of cells harboring SREBF1 knockout were collected. (A) The mRNA level of SREPF1 and its targets genes (ACACA, FASN, SCD, ACLY) were measured by RT-PCR. (B) Determine FASN, ACC and SCD1 in MCF7 cells with SREBF1 knockout by western blot.

FIG. 11 A-H. SCD1 protects cells against ferroptosis. (A) Cells were pretreated cells with 5 μM CAY10566 overnight. Cells were treated as indicated. RSL3, 1 μM for MDA-MB-453 and 0.5 μM for BT474; Fer-1, 1 μM; CAY10566, 5 μM; Fer-1, 1 μM. (B) Western blot, measuring the SCD knockout efficiency in BT474, MDA-MB-453 and MCF7 cells. (C) MCF cells (sgCtrl, sgSCD #1 and sgSCD #2) were treated as indicated. RSL3, 5 μM; Fer-1, 1 μM. (D) Cells were treated as indicated. RSL3, 1 μM for MDA-MB-453 cells and 0.5 μM for BT474 cells; Fer-1, 1 μM. (E) Cells were treated as indicated. RSL3, 1 μM for MDA-MB-453 cells and 0.5 μM for BT474 cells; Fer-1, 1 μM; CCI-779, 0.5 μM; GDC-0941, 2 μM; MK-2206, 2 μM. (F) SCD1 was overexpressed in BT474 cells. Cells were treated as indicated. RSL3, 0.5 μM; CCI-779, 0.5 μM. Lipid peroxidation was measured. (G) SCD1 was overexpressed in A549 cells and determined by western blot. Cells were treated as indicated. RSL3, 0.25 μM; CCI-779, 0.5 μM. Lipid peroxidation and cell death were measured 6 h and 24 h after treatment, respectively. (H) SCD1 was overexpressed in BT474 cells harboring SREBF1 knockout. SCD1 and SREBP1 level were determined by western blot. Cells were treated as indicated. RSL3, 0.5 μM; CCI-779, 0.5 μM; Fer-1, 1 μM.

FIG. 12 A-C. Ferroptosis sensitization triggered by mTORC1 inhibition can be prevented by exogenous MUFAs. (A) An overview of lipogenesis regulated by SREBP1-driven transcription. (B) A549 cells were treated as indicated. Oleic acid (18:1, OA), 0.5 mM; stearic acid (18:0, SA), 0.5 mM; RSL3, 0.5 μM; CCI-779, 0.5 μM. (C) Cells were treated as indicated. Palmitoleic acid (16:1, PO), 0.5 mM; Palmitic acid (16:0, PA), 0.5 mM; RSL3, 1 μM for MDA-MB-453 cells and 0.5 μM for BT474 cells; CCI-779, 0.5 μM.

FIG. 13 A-E. Combination of mTORC1 inhibition with ferroptosis induction leads to tumor regression. (A) GPX4-iKO BT474 cells were treated as indicated for 30 h. CCI-779, 0.5 μM; DOX, 100 ng/ml; Trolox, 200 μM. Dead cells were stained with Sytox Green (scale bar, 100 m). (B) BT474 tumor volume was measured every day for each mouse. The log 2 fold change of tumor volume of each individual mouse was plotted. (C) Images of resected tumors from mice xenografted with PC-3 cells. Groups of mice were treated with CCI-779 and/or IKE as indicated (n=6 per group). See Methods for detail. (D) Representative haematoxylin and eosin (H&E) and immunostaining images of Ki67, PTGS2 and pS235/236 S6, all counterstained with haematoxylin (blue), are shown from sections of PC-3 xenografted tumors. Scale bar, 50 μm. (E) PC-3 tumor volume was measured every day for each mouse. The log 2 fold change of tumor volume of each individual mouse was plotted.

### DETAILED DESCRIPTION

While some of the main embodiments of the present invention are described in the Summary of the Invention, Examples and Claims sections of this patent disclosure, this Detailed Description section provides certain additional description relating to the invention and is intended to be read in conjunction with all other sections of the present patent application.

Unless defined otherwise, all technical and scientific terms used herein have the same meaning as commonly understood to one of ordinary skill in the art to which this invention belongs.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges provided herein are inclusive of the numbers defining the range.

Where a numeric term is preceded by “about” or “approximately,” the term includes the stated number and values ±10% of the stated number. Furthermore, whenever a numeric term is preceded by the qualifier “about,” an alternative embodiment having the precise stated numeric value without the “about” qualifier is also contemplated and also falls within the scope of the present invention. Conversely, whenever an embodiment of the present invention refers to a specific numeric term, an alternative embodiment with an “about” qualification is also contemplated and also falls within the scope of the present invention.

The terms “comprising,” “consisting of”, and/or “consisting essentially of” have their accepted meanings according to U.S. patent law. Wherever embodiments are described with the language “comprising,” otherwise analogous embodiments described in terms of “consisting of” and/or “consisting essentially of” are included.

### I. Active Agents & Compositions

The methods and compositions provided by present invention involve various different active agents, including, but not limited to, the following:

Inhibitors of the PI3K/Akt/MTOR signaling axis, including, but not limited to, PI3K inhibitors, Akt inhibitors, MTOR inhibitors, MTORC1 inhibitors, SREBP inhibitors and SCD1 inhibitors;

PI3K inhibitors, including, but not limited to, GDC-0941, SAR245409, SAR245408, BYL-719, GDC-0980, wortmannin, Ly294002, demethoxyviridin, perifosine, delalisib, idelaisib, PX-866, IPI-145, BAY 80-6946, BEZ235, RP6530, TGR 1202, RP5264, SF1126, INK1117, BKM120, Palomid 529, GSK1059615, ZSTK474, PWT33597, IC87114, TG100-115, CAL263, RP6503, PI-103, GNE-477, CUDC-907 and AEZS-136;

Akt inhibitors, including, but not limited to, MK-2206, MK-2206, perifosine, GSK690693, ipatasertib (GDC-0068), AZD5365, afuresertib (GSK2110183), At13148, PF-04691502, AT7867, triciribine, CCT128930, A-674563, PHT0427, miltefosine, honokiol, and TIC10;

MTOR inhibitors, including, but not limited to, SAR245409, GDC-0980, CCI-779, KU-0063794, rapamycin, epigallocatechin gallate (EGCG), caffeine, curcumin, resveratrol, sirolimus, temsirolimus, everolimus, and ridaforolimus;

MTORC1 inhibitors, including, but not limited to, Temsirolimus (CCI-779), Torin and a short hairpin RNA (shRNA) inhibitor of RAPTOR;

SREBP inhibitors, including, but not limited to, Fatostatin A;

SCD1 inhibitors, including, but not limited to, CAY10566; and

Ferroptosis inducers, including, but not limited to, RSL3, erastin, imidazole ketone erastin (IKE), sulfasalazine, sorafenib, altretamine, artesunate, ML-162 and ML-210.

In some embodiments the present invention provides therapeutic compositions comprising one or more of the active agents described herein and a therapeutically acceptable carrier. A “therapeutically acceptable carrier” is, or comprises, a substance that is useful in preparing a composition suitable for administration to a living subject (such as a living human subject) and that is generally safe and non-toxic. Suitable “therapeutically acceptable carriers” may be, or may comprise, a saline solution (e.g., a phosphate buffered saline solution), water, an emulsion (such as an oil/water or water/oil emulsion), a wetting agent, a diluent, a filler, a salt, a buffer, a stabilizer, a solubilizer, a lipid, or any other substance known in the art for use in preparing a composition suitable for administration to a living subject.

### II. Methods of Treatment

The present invention provides various methods of treatment. As used herein, the terms “treat,” “treating,” and “treatment” refer to improving (or to methods that improve), to a detectable degree, one or more clinical indicators or symptoms associated with a tumor (such as a tumor of a specified type). For example, such terms include, but are not limited to, reducing the rate of growth of a tumor (or of tumor cells), halting the growth of a tumor (or of tumor cells), causing regression of a tumor (or of tumor cells), reducing the size of a tumor (for example as measured in terms of tumor volume or tumor mass), reducing the grade of a tumor, eliminating a tumor (or tumor cells), and the like. The efficacy of a given composition or method in treatment can be demonstrated or assessed using standard methods known in the art, such as methods that compare the efficacy of a given/“test” composition or method to a “control” composition or method. For example, the efficacy of a given composition or method in treating a tumor may be demonstrated or assessed by comparing its ability to improve one or more clinical indicators or symptoms of a tumor as compared to that of a control composition or control method, such as a placebo control. For example, a comparison can be made between different subjects (e.g., between a test group of subjects or a control group of subjects). Similarly, the efficacy of a given composition or method in treatment can be demonstrated or assessed in a single subject by comparing that subject's tumor before and after treatment.

The term “tumor” is used herein in accordance with its normal usage in the art and includes a variety of different tumor types.

In carrying out the treatment methods described herein, any suitable method or route of administration can be used to deliver the active agents or combinations thereof described herein. The term “administration” includes any route of introducing or delivering the specified compositions or agents to subjects. In some embodiments the active agents or combinations thereof, are administered systemically. In some embodiments the active agents or combinations thereof, are administered locally. “Systemic administration” refers to introducing or delivering to a subject a specified composition or agent via a route which introduces or delivers the composition or agent to extensive areas of the subject's body (e.g., greater than 50% of the body), for example through entrance into the circulatory or lymph systems. By contrast, “local administration” refers to introducing or delivering to a subject a specified composition or agents via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body.

In some embodiments administration can be carried out by any suitable route known in the art, including intratumoral, intravenous, subcutaneous, oral, topical, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. Administration includes self-administration and administration by another. The suitability of a given route or means of administration can be readily determined by a physician.

Where two or more active agents are administered, the agents may be administered simultaneously, sequentially, or at overlapping times. Similarly, where two or more agents are administered, the agents may be administered together in the same composition or separately in different compositions.

As used herein the term “effective amount” refers to an amount of an active agent as described herein that is sufficient to achieve, or contribute towards achieving, one or more of the outcomes listed in the “treatment” description herein. An appropriate “effective” amount in any individual case may be determined using standard techniques known in the art, such as dose escalation studies, and may be determined taking into account such factors as the desired route of administration (e.g., systemic vs. local), the desired frequency of dosing, etc. Furthermore, an “effective amount” may be determined in the context of any co-administration to be used. One of skill in the art can readily perform such dosing studies (whether using single agents or combinations of agents) to determine appropriate doses to use, for example using assays such as those described in the Examples section of this patent application—which involve administration of the agents described herein to subjects—such as animal subjects routinely used in the pharmaceutical sciences for performing dosing studies.

For example, in some embodiments the dose of an active agent of the invention may be calculated based on studies in humans or other mammals carried out to determine efficacy and/or effective amounts of the active agent. The dose amount and frequency or timing of administration may be determined by methods known in the art and may depend on factors such as pharmaceutical form of the active agent, route of administration, whether only one active agent is used or multiple active agents (for example, the dosage of a first active agent required may be lower when such agent is used in combination with a second active agent), and patient characteristics including age, body weight or the presence of any medical conditions affecting drug metabolism.

In those embodiments described herein that refer to specific doses of agents to be administered based on mouse studies, one of skill in the art can readily determine comparable doses for human studies based on the mouse doses, for example using the types of dosing studies and calculations described herein.

In some embodiments suitable doses of the various active agents described herein can be determined by performing dosing studies of the type that are standard in the art, such as dose escalation studies, for example using the dosages shown to be effective in mice in the Examples section of this patent application as a starting point.

Dosing regimens can also be adjusted and optimized by performing studies of the type that are standard in the art, for example using the dosing regimens shown to be effective in mice in the Examples section of this patent application as a starting point. In some embodiments the active agents are administered daily, or twice per week, or weekly, or every two weeks, or monthly.

In certain embodiments the compositions and methods of treatment provided herein may be employed together with other compositions and treatment methods known to be useful for tumor therapy, including, but not limited to, surgical methods (e.g., for tumor resection), radiation therapy methods, treatment with chemotherapeutic agents, treatment with antibodies, treatment with immunotherapeutic agents, treatment with cell therapy methods, treatment with tyrosine kinase inhibitors, and the like. Similarly, in certain embodiments the methods of treatment provided herein may be employed together with procedures used to monitor disease status/progression, such as biopsy methods and diagnostic methods (e.g., MRI methods or other imaging methods).

For example, in some embodiments the methods described herein and/or the agents and compositions described herein may be employed or administered to a subject prior to performing surgical resection of a tumor, for example in order to shrink a tumor prior to surgical resection. In other embodiments the methods described herein and/or the agents and compositions described herein may be employed or administered to a subject both before and after performing surgical resection of a tumor.

### III. Subjects

As used herein the term “subject” encompasses all mammalian species, including, but not limited to, humans, non-human primates, dogs, cats, rodents (such as rats, mice and guinea pigs), cows, pigs, sheep, goats, horses, and the like—including all mammalian animal species used in animal husbandry, as well as animals kept as pets and in zoos, etc. In some embodiments the subjects are human. Such subjects will typically have a tumor (or tumors) in need of treatment.

The invention is further described in the following non-limiting Example, as well as the Figures referred to therein.

### Example

Ferroptosis, a form of regulated necrosis driven by iron-dependent peroxidation of phospholipids, is regulated by cellular metabolism, redox homeostasis, and various signaling pathways related to cancer. In this study, we found that activating mutation of PI3K or loss of PTEN function, highly frequent events in human cancer, confers ferroptosis resistance in cancer cells, and that inhibition of the PI3K-AKT-mTOR signaling axis sensitizes cancer cells to ferroptosis induction. Mechanistically, this resistance requires sustained activation of mTORC1 and the mTORC1-dependent induction of sterol regulatory element-binding protein 1 (SREBP1), a central transcription factor regulating lipid metabolism. Furthermore, stearoyl-CoA desaturase-1 (SCD1), a transcriptional target of SREBP1, mediates the ferroptosis-suppressing activity of SREBP1 by producing monounsaturated fatty acids. Genetic or pharmacologic ablation of SREBP1 or SCD1 sensitized ferroptosis in cancer cells with PI3K-AKT-mTOR pathway mutation. Conversely, ectopic expression of SREPB1 or SCD1 restored ferroptosis resistance in these cells, even when mTORC1 was inhibited. In xenograft mouse models for PI3K-mutated breast cancer and PTEN-defective prostate cancer, the combination of mTORC1 inhibition with ferroptosis induction resulted in near-complete tumor regression. In conclusion, hyperactive mutation of PI3K-AKT-mTOR signaling protects cancer cells from oxidative stress and ferroptotic death through SREBP1/SCD1-mediated lipogenesis, and combination of mTORC1 inhibition with ferroptosis induction shows therapeutic promise in preclinical models.

### Results

Oncogenic activation of the PI3K-AKT-mTOR signaling pathway confers resistance to ferroptosis

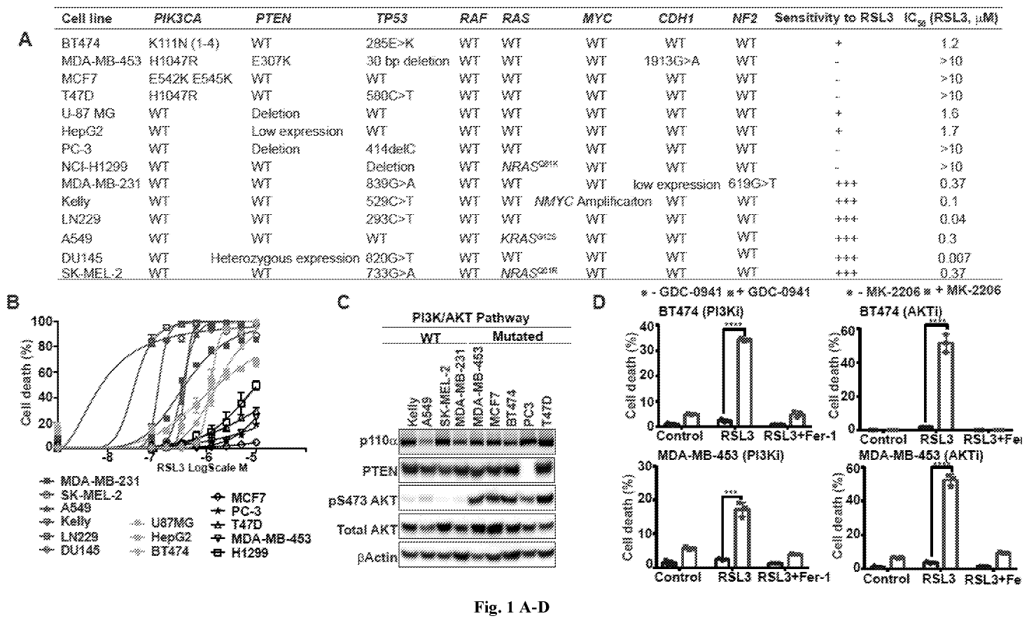

To investigate the functional interplay between ferroptosis and signaling pathways frequently mutated in cancer, we analyzed a panel of human cancer cell lines with defined genetic mutations. Ferroptosis was triggered by RSL3, a pharmacological inhibitor of GPX4. The sensitivity to ferroptosis induction varied among tested lines. Notably, we found that cancer cells carrying PIK3CA activating mutation or PTEN deletion appeared to be more resistant to RSL3 (FIG. 1, A-C). These mutations lead to the activation of the oncogenic PI3K-AKT signaling pathway, which is one of the most frequently altered signaling pathways in human cancers (26-28).

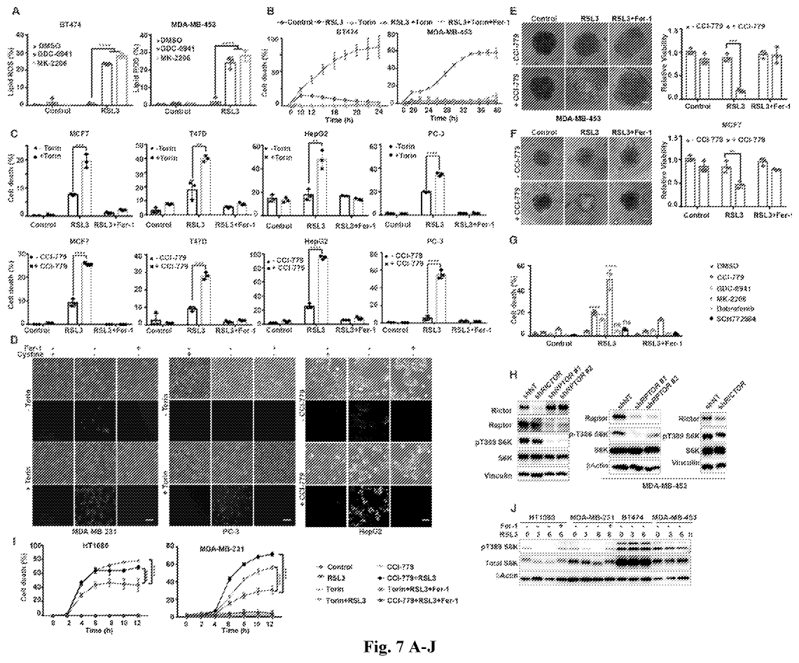

To determine whether the resistance to ferroptosis is a result of PI3K-AKT signaling pathway activation, we tested whether pharmacological inhibition of this pathway could sensitize cancer cells to ferroptosis induction. Indeed, both the PI3K inhibitor (PI3Ki) GDC-0941 and AKT inhibitor (AKTi) MK-2206 sensitized BT474 and MDA-MB-453 cells (both harboring activating mutation of the pathway) to ferroptosis (FIG. 1D) and lipid peroxidation (FIG. 7A). Moreover, inhibition of mTOR, a major downstream player of the PI3K-AKT pathway, by its catalytic inhibitor Torin also sensitized these cells to ferroptosis induced by RSL3 (FIG. 7B). Therefore, sustained activation of PI3K-AKT-mTOR pathway protects cancer cells from ferroptotic cell death. Notably, a role of mTORC1 in ferroptosis inhibition has been reported previously, such as in the context of cardiomyocytes (29), but the underlying mechanisms remain elusive.

mTORC1, Instead of mTORC2, Suppresses Ferroptosis

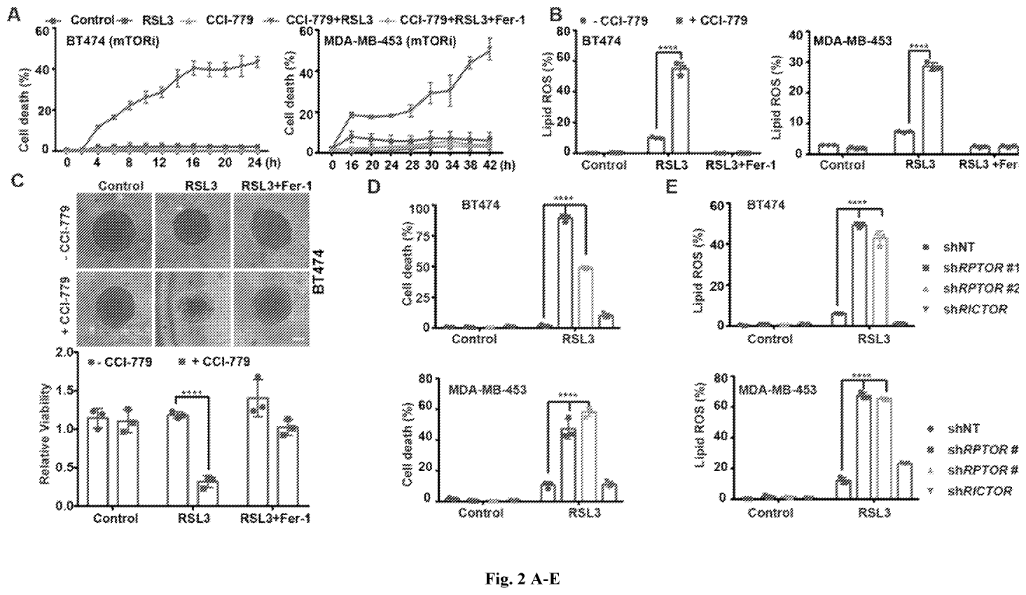

mTOR signaling is mediated by two branches, mTORC1 and mTORC2 (28). To determine which branch is responsible for the observed ferroptosis regulation, we first tested rapalog Temsirolimus (CCI-779), which inhibits the activity of mTORC1 but not that of mTORC2. Similar to Torin, CCI-779 could sensitize cancer cells to ferroptosis induction and lipid peroxidation (FIG. 2, A-B and FIG. 7, C-D). Consistently, in a three-dimensional (3D) tumor spheroid system, mTOR inhibition also synergized with RSL3 in inducing ferroptosis in these mutant cancer cells (FIG. 2C and FIG. 7, E-F). In contrast, inhibitors of ERK or BRAF failed to do so (FIG. 7G).

As both rapalog CCI-779 and mTOR catalytic inhibitor Torin can restore ferroptosis sensitivity, we hypothesized that mTORC1 instead of mTORC2 is responsible for the resistance of cancer cells with PI3K-AKT pathway mutation. Consistent with this notion, short hairpin RNA (shRNA)-mediated silencing of RAPTOR (a component of mTORC1) but not that of RICTOR (a component of mTORC2) sensitized MDA-MB-453 and BT474 cells to RSL3 (FIG. 2, D-E and FIG. 7H).

We next examined cancer cells that express wild-type PI3K-AKT-mTOR pathway and are sensitive to ferroptosis induction. We tested in these wild-type cells how basal activity of the pathway allowed ferroptosis induction, and whether pharmacological inhibition of the pathway could further enhance ferroptosis. We used two cell lines with wild-type PI3K-AKT-mTOR pathway, HT1080 and MDA-MB-231. Although low concentration of RSL3 alone was sufficient to induce ferroptosis in these cells, addition of mTOR inhibitors further enhanced ferroptosis (FIG. 7I). Notably, mTORC1 activity was inhibited by RSL3 over time in these wild-type cells, as measured by S6K phosphorylation (FIG. 7J). In contrast, cells harboring pathway mutation retained active mTORC1 upon RSL3 treatment for the same time period (FIG. 7J). Interestingly, treatment with the lipid peroxide-trapping agent ferrostatin-1 (Fer-1) prevented RSL3-triggered inactivation of mTORC1 activity in wild-type cells (FIG. 7J), suggesting lipid peroxidation is responsible for, and precedes, mTORC1 inactivation in response to RSL3. However, in cancer cells harboring PI3K-AKT-mTOR pathway mutation, RSL3-induced lipid peroxidation became clear only when the pathway was inhibited (FIG. 7A and FIG. 2), suggesting mTOR inhibition is responsible for substantial accumulation of lipid peroxides in these mutant cells. These results indicate that mTORC1 activity acts to prevent the toxic effect of lipid peroxides, whereas the accumulation of ROS and lipid peroxides in cells attenuates mTORC1 activity. In such a way, upon ferroptosis induction, lower basal mTORC1 activity in wild-type cells allows lipid peroxide accumulation, which in turn leads to the inhibition of mTORC1 activity and accelerated lipid peroxidation; but in mutant cancer cells, the more potent and sustained mTORC1 activity prevents lipid peroxide accumulation, thus causing resistance to ferroptosis.

NRF2 is not the Major Mediator of the Ferroptosis-Suppressing Activity of mTORC1

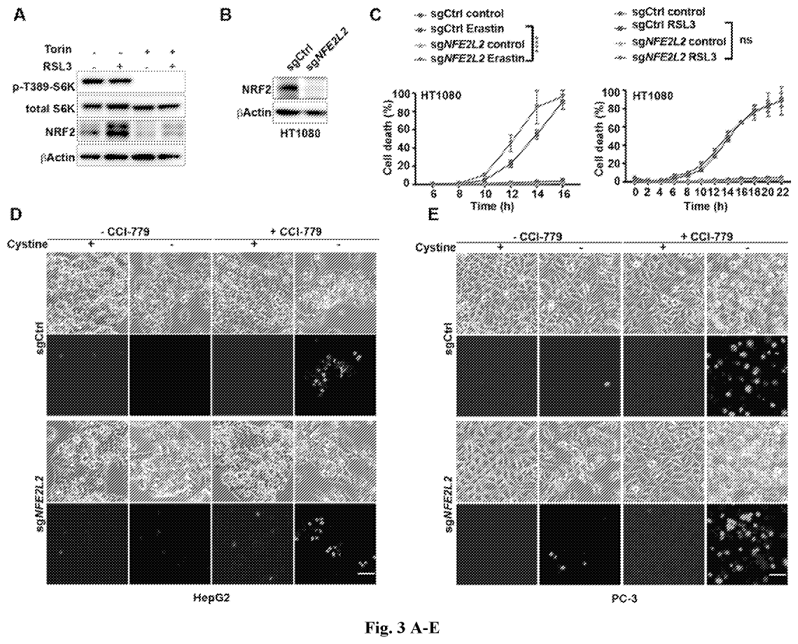

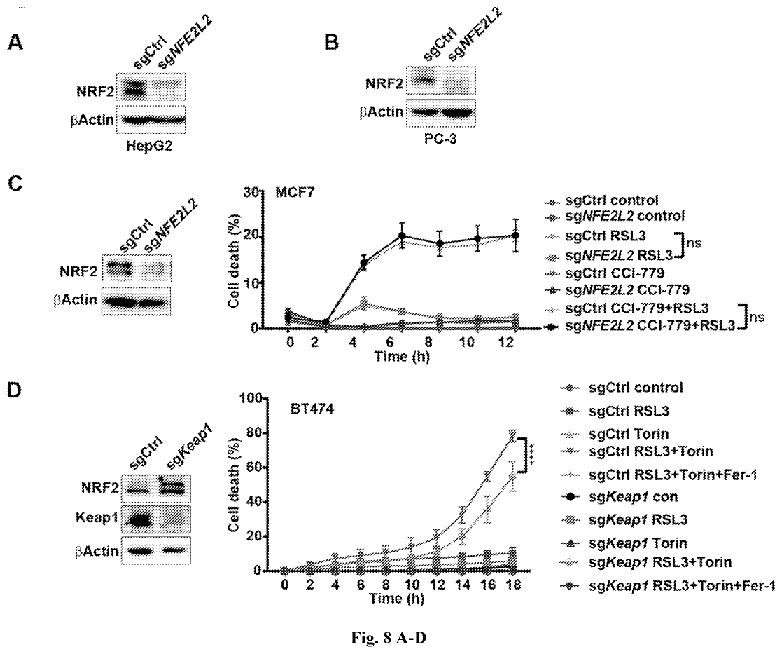

High levels of cellular ROS, a feature of ferroptosis (1), will trigger antioxidant pathway by suppressing Keap1-mediated proteasomal degradation of the NRF2 transcription factor (30). It has been reported that mTORC1 promotes the association of p62 with Keap1 by phosphorylating p62, leading to the degradation of Keap1 and hence NRF2 accumulation (31). This p62-Keap1-NRF2 axis was reported to protect hepatocellular carcinoma cells from ferroptosis (32). Furthermore, NRF2 signaling has also been suggested to mediate the effect of mTOR on ferroptosis inhibition (33). However, although we found that RSL3-induced NRF2 accumulation was ablated by Torin treatment (FIG. 3A), NRF2-knockout in HT1080 cells only modestly enhanced ferroptosis induced by erastin (a chemical inhibitor of system xc-cysteine/glutamate antiporter) and had no measurable effect on RSL3-induced ferroptosis (FIGS. 3, B and C); in multiple cell lines with PI3K pathway mutation, ferroptosis induction could still be potently sensitized by mTOR inhibitors even after NRF2 was knocked out (FIG. 3, D-E and FIG. 8, A-C). Further, Keap1 knockout and consequent NRF2 accumulation in BT474 cells only resulted in a modest reduction of ferroptosis sensitization triggered by mTORC1 inhibition (FIG. 8D). These results indicate that ferroptosis sensitization by mTORC1 inhibition is mainly through NRF2-independent mechanisms.

mTORC1 Activation Suppresses Ferroptosis by Upregulating SREBP1.

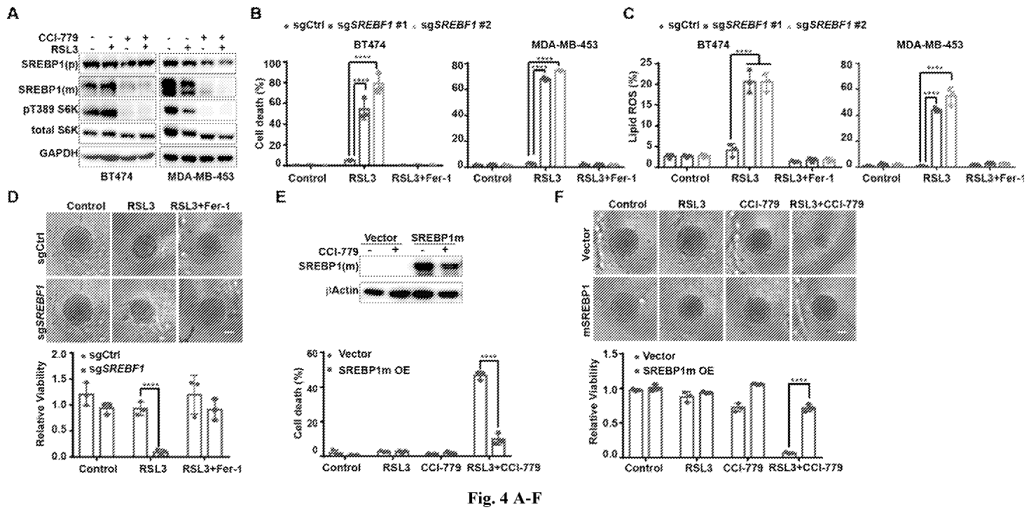

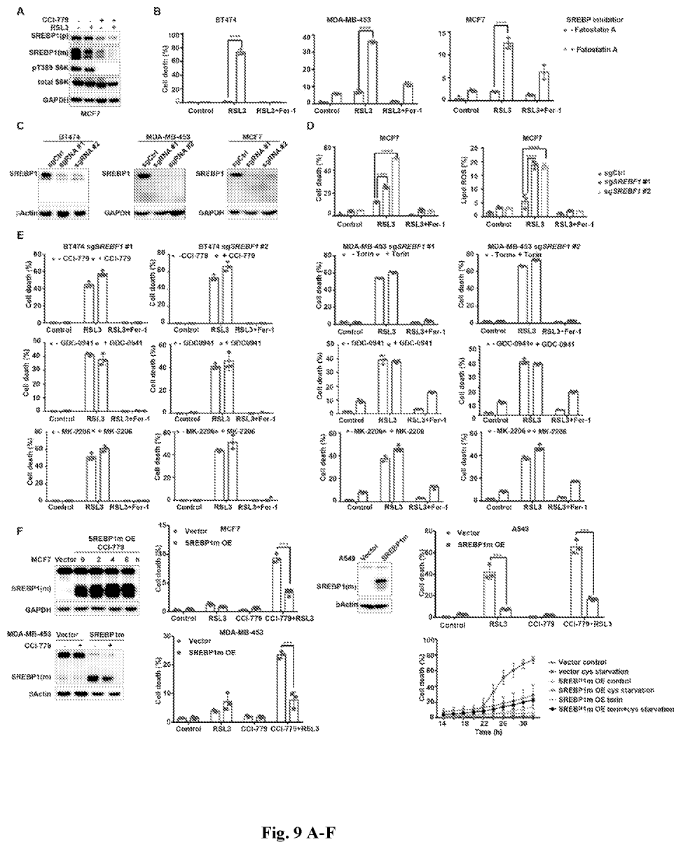

Ferroptotic cell death requires phospholipid peroxidation. To investigate if mTORC1 regulates ferroptosis through modulating cellular lipid metabolism, we examined a central regulator of lipid synthesis, SREBP1(20, 21), which was recently demonstrated as a downstream target of mTORC1 activity (22, 25, 34, 35). Indeed, in multiple types of RSL3-resistant cancer cells, mTORC1 inhibitor CCI-779 decreased the level of the mature form of SREBP1 (SREBP1m) that can translocate into the nucleus to regulate its downstream transcriptional targets (FIG. 4A, FIG. 9A). Functionally, pharmacological inhibition of SREBP activity by Fatostatin A (36) or genetic deletion of the SREBF1 gene by CRISPR/cas9 sensitized ferroptosis and lipid peroxidation in these cells (FIG. 4, B-D and FIG. 9, B-D). Moreover, in these SREBF1-knockout cells, mTOR inhibitors (Torin and CCI-779), PI3K inhibitor (GDC-0941) and AKT inhibitor (MK-2206) all failed to further enhance RSL3-induced ferroptosis (FIG. 9E). Conversely, ectopic expression of the constitutively active nuclear form of SREBP1 (SREPB1m), as test in both 2D cell culture and 3D tumor spheroid experiments, rendered (1) the otherwise RSL3-sensitive A549 cells resistant, and (2) multiple RSL3-resistant cell lines not responsive any longer to the combination of CCI-779 with RSL3 (FIG. 4, E-F and FIG. 9F). In conclusion, mTORC1 promotes cancer cell resistance to ferroptosis induction through the upregulation of SREBP1 function.

SREBP1 Protects Cells from Ferroptosis Through SCD1 Activity.

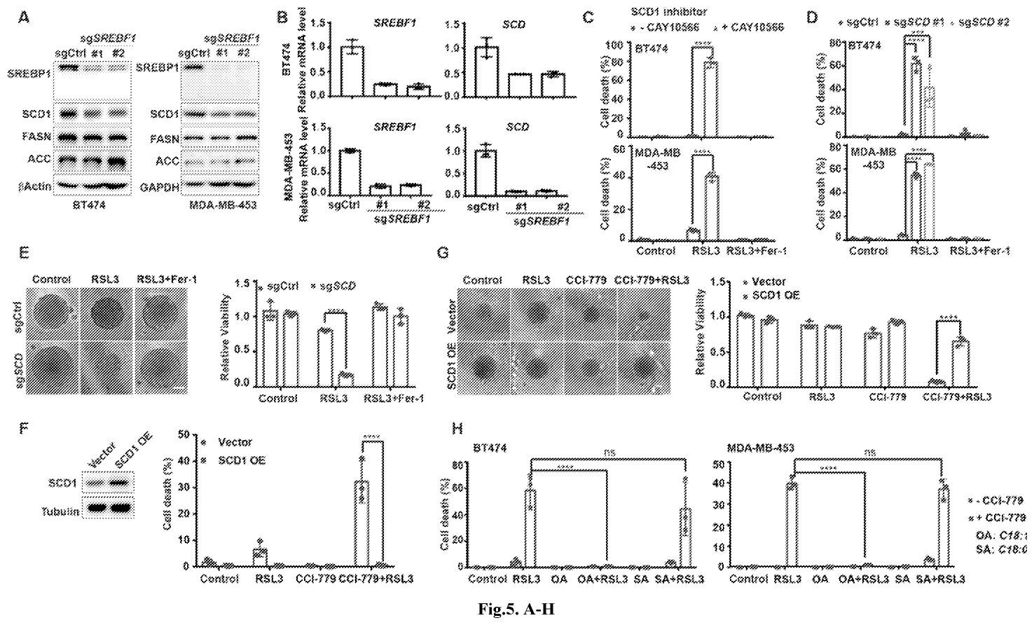

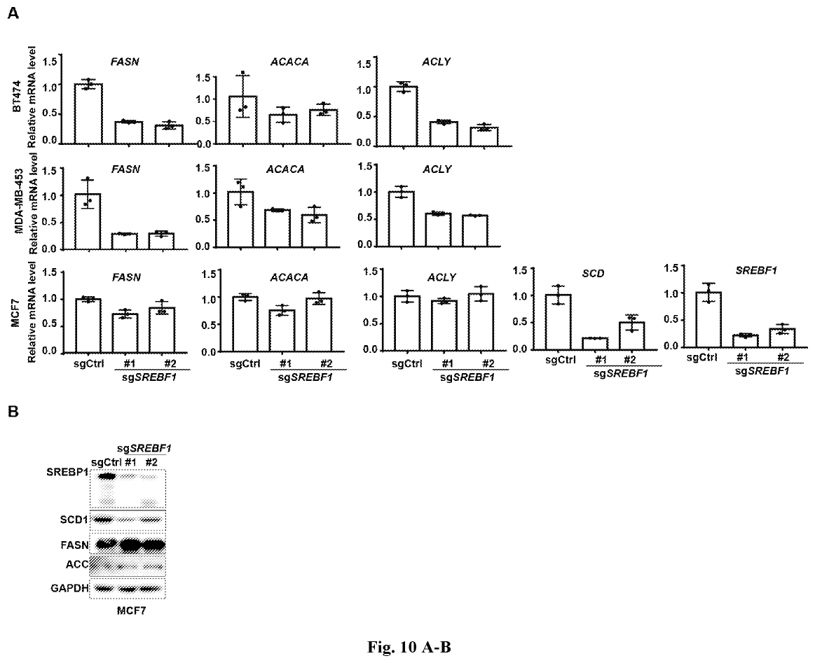

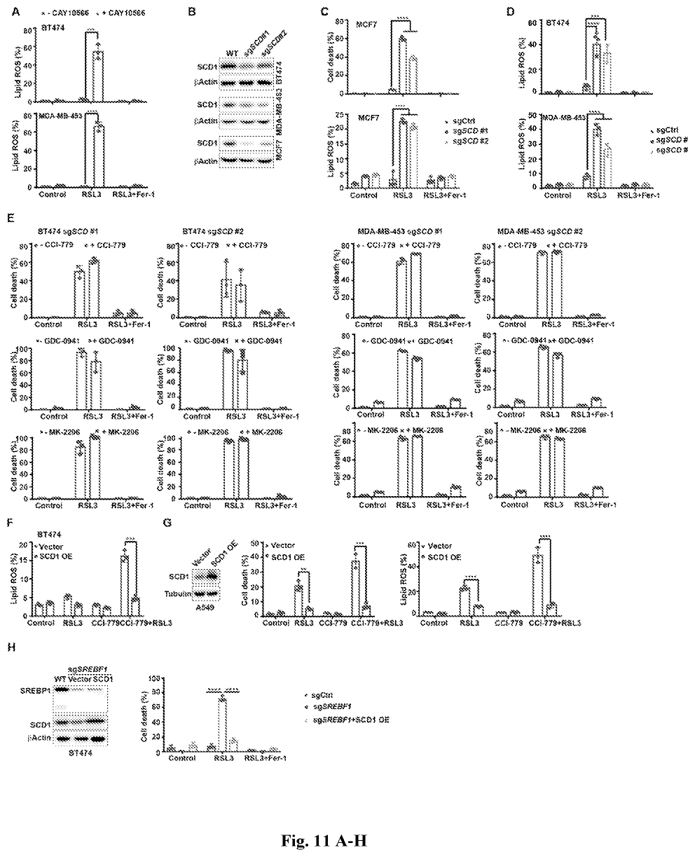

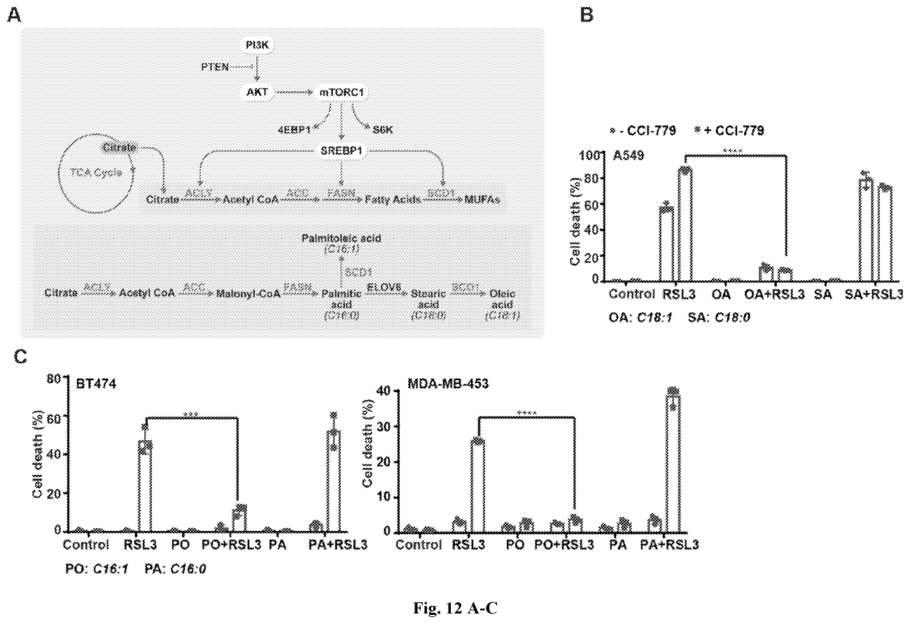

SREBP1 is a transcription factor that regulates, among other metabolic genes, multiple lipid synthesis-related genes including ACLY, ACACA, FASN, and SCD (FIG. 12A)(20). In the tested cell lines bearing PI3K-AKT-mTOR pathway mutation, SREBF1 knockout decreased the expression of SCD1 (both mRNA level and protein level) more significantly than that of other targets (FIG. 5, A-B and FIG. 10). This result and the recently reported anti-ferroptotic function of SCD1(37) prompted us to examine whether SCD1 is the major downstream target of SREBP1 that mediates the resistance to ferroptosis induction. Pharmacologically, SCD1 inhibitor CAY10566 sensitized the effect of RSL3 on the induction of ferroptosis (FIG. 5C) and lipid peroxidation (FIG. 11A). Genetically, CRISPR/Cas9-mediated SCD knockout also sensitized cells to ferroptosis induction and lipid peroxidation (FIG. 5, D-E and FIG. 11, B-D). Further, upon SCD knockout, inhibition of mTORC1, PI3K, or AKT could not further sensitize cancer cells to ferroptosis (FIG. 11E). Conversely, SCD1 overexpression protected cancer cells from ferroptosis induced by the combination of RSL3 with mTOR inhibition or with SREBF1 knockout (FIG. 5, F-G and FIG. 11, F-H).

SCD1 is an enzyme that converts saturated fatty acids to monounsaturated fatty acids (MUFAs) (FIG. 12A). It has been reported that MUFAs can inhibit ferroptosis (38), providing a mechanistic explanation to our observation. Indeed, supplementation of MUFA palmitoleic acid (16:1, PO) or oleate acid (18:1, OA), but not saturated fatty acid palmitic acid (16:0, PA) or stearic acid (18:0, SA), resulted in ferroptosis resistance upon treatment of CCI-779 plus RSL3 (FIG. 5H and FIG. 12, B-C). Collectively, these results indicate that SREBP1 protects cancer cells from ferroptosis mainly by upregulating SCD1. Remarkably, SCD1 is an iron-dependent enzyme that catalyzes fatty acid desaturation, which is by nature an oxidative reaction; and we found here that this iron-dependent, oxidative enzymatic reaction can mitigate ferroptosis, an iron-dependent, oxidative form of cell death.

Combination of mTORC1 Inhibition with Ferroptosis Induction Leads to Tumor Regression In Vivo

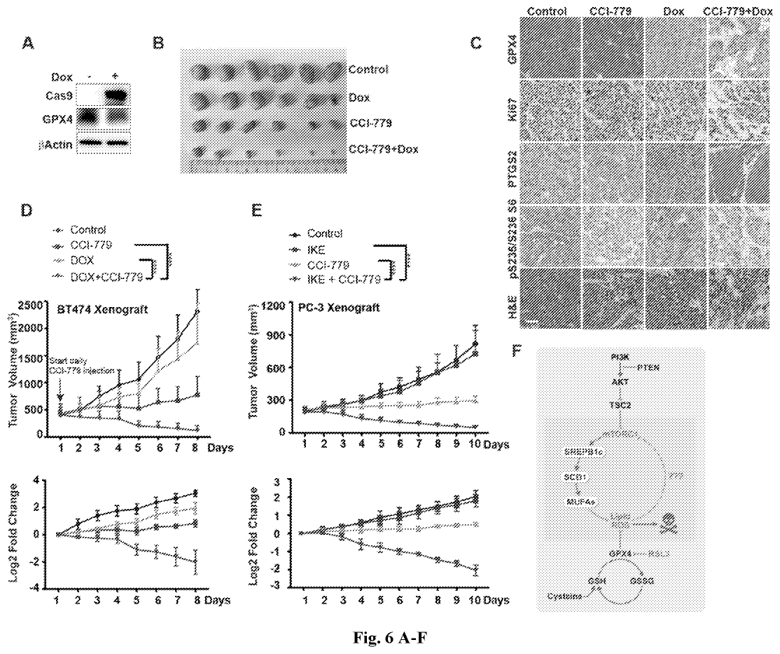

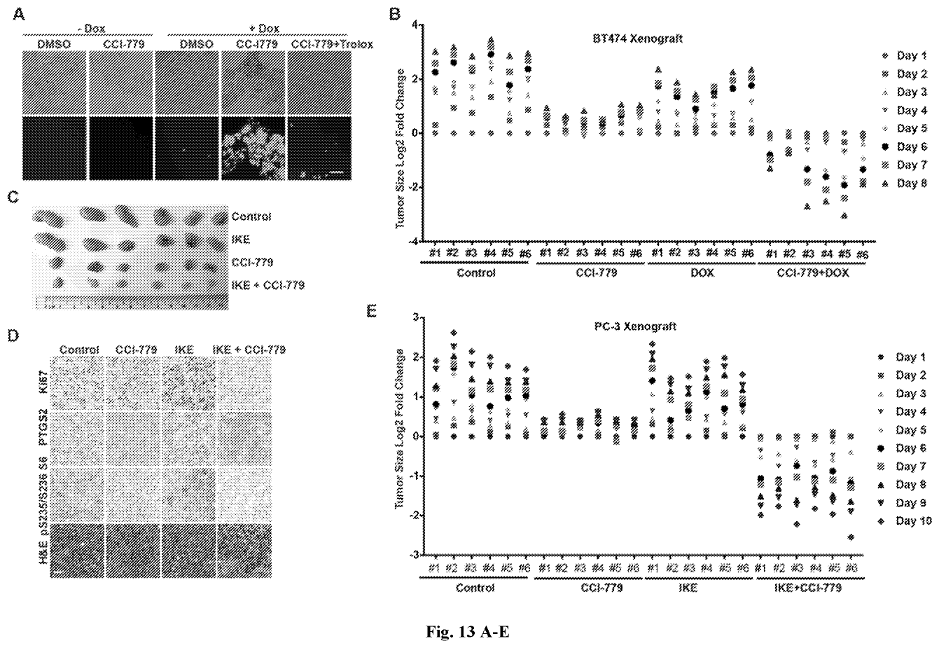

To explore the cancer therapeutic potential of combining mTORC1 inhibition with ferroptosis induction, we analyzed two mouse xenograft models for human cancer. In the first model, we generated CRISPR/Cas9-mediated GPX4 knockout in a doxycycline (Dox)-inducible manner in PI3K-mutated BT474 breast cancer cells (FIG. 6A). In these cells, only the combination of GPX4 knockout with mTORC1 inhibition, but not either alone, induced potent ferroptosis (FIG. 13A). In mice xenografted with these cells, we allowed the average volume of tumors to reach ˜400 mm3, and then started mTORC1 inhibition by CCI-779 administration (Dox administration was started two days earlier). While CCI-779 administration decelerated tumor growth, strikingly, the combination of Dox treatment with CCI-779 caused a near-complete regression of tumors (FIG. 6B, 6D and FIG. 13B). Immunohistochemical analysis of PTGS2, a marker of oxidative stress and ferroptosis(3), supported such synergistic effect of the combining inhibition of GPX4 and mTORC1 in inducing tumor ferroptosis in vivo (FIG. 6C). In the other mouse model, we used imidazole ketone erastin (IKE), a potent and metabolically stable analog of erastin that has been validated for in vivo use(39), instead of genetic deletion of GPX4, to induce tumor cell ferroptosis. PTEN-defective PC-3 prostate cancer cells were used to generate xenograft tumors in mice (PTEN deficiency predicts poor prognosis in prostate cancer (40)). Similar to what we observed in BT474 xenograft experiment, here IKE alone has no effects on tumor growth, but its combination with CCI-779 resulted in dramatic tumor regression (FIG. 6E and FIG. 13, C-E). Collectively, these two in vivo experiments demonstrate that the combination of mTORC1 inhibition with ferroptosis induction is a promising therapeutic approach for the treatment of cancer harboring activating mutation of the PI3K-AKT-mTORC1 pathway.

### DISCUSSION

In conclusion, this work reveals a novel ferroptosis-regulatory mechanism in that oncogenic activation of the PI3K-AKT-mTORC1 pathway suppresses ferroptosis in cancer cells via downstream SREBP1/SCD1-mediated lipogenesis (FIG. 6F). Previously, it has been reported that various metabolic processes, such as glutaminolysis and mitochondrial TCA cycle (11, 41), contribute to ferroptosis. This study unravels the function of SREBP-mediated lipid metabolism in ferroptosis regulation. Therefore, cellular metabolism, including that of amino acids, carbohydrates, and lipids, can all regulate ferroptosis. Cancer cells usually endure elevated oxidative stress due to increased metabolic and proliferative burden (42, 43). Consequently, pathways countering oxidative stress, particularly NRF2 signaling (30), are often activated in cancer cells. This work shows that mTORC1 is also an important modulator of cellular redox homeostasis, and it does so through both NRF2-mediated signaling and SREBP1/SCD1-mediated WUFA synthesis. The latter mechanism is crucial for the suppression of a probably more devastating type of oxidative stress, ferroptosis-inducing lipid peroxidation. It should be noted that the precise mechanism underlying the ferroptosis-inhibitory activity of MUFA has not been defined, although this activity has been demonstrated previously (38) and is responsible for the role of the oncogenic PI3K-AKT-mTORC1 pathway in suppressing ferroptosis.

Relevant to cancer, this study indicates that oncogenic alterations in PI3K-AKT-mTOR signaling, one of the most mutated pathways in human cancer (26-28), render cancer cells more resistant to ferroptosis induction. Intriguingly, it has been reported recently that oncogenic mutations in the E cadherin-NF2-Hippo-YAP pathway, also a frequently mutated signaling pathway in human cancer, instead sensitize cancer cells to ferroptosis (18). Therefore, whether and how a specific cancer-driving mutation enhances or suppresses cancer cell ferroptosis depends on how the gene product regulates cellular processes related to ferroptosis, such as metabolism and redox and iron homeostasis. In either case, these mechanistic findings provide highly valuable insights into future ferroptosis-inducing cancer therapies: malignant mutations in the E cadherin-NF2-Hippo-YAP pathway can be used as biomarkers that predict cancer cell responsiveness to ferroptosis induction as a monotherapy; and patients bearing tumorigenic mutations in the PI3K-AKT-mTOR pathway might be treated effectively by therapies combining ferroptosis induction with inhibitors of mTORC1 or other components of the pathway, many of which are available clinically. As both pathways are highly mutated in various types of malignancy, ferroptosis induction holds enormous potential for cancer treatment.

### Materials and Methods

### Reagents Used

RSL3 (1219810-16-8, Cayman), Torin (10997, Cayman), Temsirolimus (CCI-779, NSC 683864, Selleck), Ferostatin-1 (17729, Caymen), MK-2206 (S1078, Selleck Chemicals), GDC-0941 (S1065, Selleck Chemicals), CAY10566(10012562, Cayman Chemicals), Fatostatin A (4444, Tocris), SYTOX Green (S7020, Thermo Fisher, Waltham, MA, USA), propidium iodide (556463, BD Biosciences, San Jose, CA, USA), BODIPY 581/591 C11 (Thermo Fisher, Cat #D3861), Oleic acid (01383, Sigma-Aldrich), Stearic acid (S4751, Sigma), Palmitic acid (P0500, Sigma-Aldrich), Palmitoleic acid (P9417, Sigma), Imidazole ketone erastin (IKE, HY-114481, MCE).

### Cell Culture

Kelly was obtained from Sigma-Adlrich. MEF, HT1080, MDA-MB-231, MDA-MB-453, BT474, MCF7, T47D, U87MG, HepG2, PC-3, DU145, A549, NCI-H1299, LN229 and SK-MEL-2 were obtained from the American Tissue Culture Collection (ATCC) and cultured in media conditions recommended by the ATCC in a humidified atmosphere containing 5% C02 at 37° C. Media was prepared by the MSKCC Media Preparation Core Facility. All cell lines were subjected to STR authentication through ATCC or MSKCC IGO Core Facility.

### Generation of Three-Dimensional Spheroids

Spheroids were generated by plating tumour cells at 103/well into U-bottom Ultra Low Adherence (ULA) 96-well plates (Corning, Tewksbury, MA, USA). Optimal three-dimensional structures were achieved by centrifugation at 600 g for 5 min followed by addition of 2.5% Matrigel (Corning). Plates were incubated for 72 h at 37° C., 5% C02, 95% humidity for formation of a single spheroid of cells. Spheroids were then treated with RSL3 in fresh medium containing Matrigel for the indicated time.

### Cell Death Quantification and Cell Viability Measurement

Cells were seeded in plates at appropriate cell density and incubated overnight at 37° C. containing 5% C02, and then subjected to treatments as described in individual experiments. Cells were stained with hoechst 33342 (0.1 μg/ml) to monitor total cell number, and with Sytox Green (5 nM) to monitor cell death. Culture plates were read by Cytation 5 at indicated time points. Percentage of cell death was calculated as Sytox Green-positive cell number over total cell number. For 3D spheroids, cell viability was determined a commercially available cell viability assay (Promega, Madison, WI, USA) following the manufacturer's instructions. Viability was calculated by normalizing ATP levels of samples to that of negative controls (spheroids in normal full media without treatment).

### Measurement of Lipid Peroxidation

Lipid peroxidation was analyzed by flow cytometry. Cells were seeded at appropriate density in a 6-well plate and grown overnight in DMEM. Cells were stained with 5 μM BODIPY C11 (Thermo Fisher, Cat #D3861) for 30 min after indicated treatment. Labeled cells were trypsinized, re-suspended in PBS plus 2% FBS, and then subjected to flow cytometry analysis.

### Western Blot

Cell lysates were resolved on SDS-PAGE gels and transferred to a nitrocellulose membranes. The membranes were incubated in 5% skim milk for 1 hour at room temperature and then incubated with primary antibodies diluted in blocking buffer at 4° C. overnight. The following primary antibodies were used: PTEN (9559L, CST), Phospho-Akt Ser473 (4060, CST), Akt (2920, CST), PActin (Sigma-Aldrich, A1978), GAPDH (SC-47724, Santa Cruz), Raptor (2280, CST), Rictor (9476, CST), Total S6K (2708, CST), Phospho-p70 S6 Kinase Thr389 (9205, CST), ATG5 (A0731, Sigma), LC3 I/II (L7543, Sigma), SREBP1(SC-13551, Santa Cruz), SCD1 (ab39969, Abcam), FASN (3180, CST), ACACA (3662, CST), NRF2 (16396-1-AP, Proteintech Group, Inc.), Keap1 (8047S, CST), GPX4 (ab125066, Abcam) and Cas9 (14697S, CST). After three washes, membranes were incubated with goat anti-mouse HRP-conjugated antibody or donkey anti-rabbit HRP-conjugated antibody (Invitrogen) at room temperature for 1 hour and subjected to chemiluminescence using Clarity™ Western ECL Substrate (Bio-Rad, Hercules, CA, USA). An Amersham Imager 600 (GE Healthcare Life Sciences, Marlborough, MA, USA) were used for the final detection.

### RT-PCR

Total RNA was prepared with the trizol reagent (Invitrogen). 20% chloroform was added to each sample. The samples were shaken vigorously for 15 seconds and incubated at room temperature for 15 min. Samples were then centrifuged at 12,000 g for 15 min at 4° C. The aqueous phase was transferred to a new tube and an equal volume of isopropanol was added. Samples were incubated at room temperature for 10 min, followed by centrifugation at 12,000 g for 10 min at 4° C. mRNA pellets were washed in 75% ethanol, dried, and resuspended in nuclease-free water. mRNA was reverse transcribed into cDNA. cDNA was amplified with by real time PCR. The PCR program was as follows: 95° C., 30 seconds; 40 cycles (for each cycle 95° C., 15 seconds; 55° C., 40 seconds). Primers were as follows:

TABLE 1
Primer Sequences

| Gene | Forward Primer 5′-3′ | Reverse Primer 5′-3′ |
|---|---|---|
| βActin | CTCTTCCAGCCTTCCTTCCT | AGCACTGTGTTGGCGTACAG |
| | SEQ ID NO. 1 | SEQ ID NO. 2 |
| SREBF1 | GCTGCTGACCGACATCGAA | GGGTGGGTCAAATAGGCCAG |
| | SEQ ID NO. 3 | SEQ ID NO. 4 |
| FASN | AACTCCAAGGACACAGTCAC | CAGCTGCTCCACGAACTCAA |
| | CAT SEQ ID NO. 5 | SEQ ID NO. 6 |
| ACACA | GGATGGGCGGAATGGTCTCT | GCCAGCCTGTCGTCCTCAAT |
| | TT SEQ ID NO. 7 | GTC SEQ ID NO. 8 |
| ACLY | AACGCCAGCGGGAGCACATC | TTGCAGGCGCCACCTCATCG |
| | SEQ ID NO. 9 | SEQ ID NO. 10 |
| SCD | CCGGACACGGTCACCCGTTG | CGCCTTGCACGCTAGCTGGT |
| | SEQ ID NO. 11 | SEQ ID NO. 12 |

Lentiviral-Mediated shRNA Interference

Lentiviral shRNA clones targeting RPTOR and RICTOR were purchased from Sigma-Aldrich. Lentiviruses were produced by the co-transfection of the lentiviral vector with the delta-VPR envelope and CMV VSV-G packaging plasmids into 293T cells using PEI. Media was changed 8 hours after transfection. The supernatant was collected 48 hours after transfection and passed through a 0.45 μm filter. Cells were incubated with infectious particles in the presence of 4 μg/ml polybrene (Sigma-Aldrich) overnight and cells were given fresh complete medium. After 48 hours, cells were placed under the appropriate antibiotic selection.

### Retroviral-Mediated Gene Overexpression

For inducible expression of SREBP1 and SCD1, cDNAs were obtained and subcloned into a modified version of a retroviral vector. Retrovirus was produced by co-transfection of the retroviral vector with gag/pol and VSV-G into 293T cells. Virus was collected and passed through a 0.45 μm filter. Infected cells were selected in medium containing hygromycin. Gene expression was induced by addition of 100 ng/ml doxycycline to culture medium.

### Inducible CRISPR/Cas9 Mediated GPX4 Knockout

A lentiviral doxycycline (DOX)-inducible pCW-Cas9 vector and a pLX-sgRNA were used for inducible gene knockout (iKO). The sgRNA sequence targeting human GPX4 is CACGCCCGATACGCTGAGTG (SEQ ID NO. 19). Lentivirus was packaged in 293T cells. Medium was changed 8 h after transfection, and the virus-containing supernatant was collected and filtered 48 h after transfection. BT474 cells in 6-well tissue culture plates were infected with pCW-Cas9 viral supernatant containing 4 μg/mL polybrene. Cells were selected with 2 μg/ml puromycin after 48 h after infection. Single clones were screened for DOX-inducible Cas9 expression. Single clones with Cas9 expression were infected with the GPX4 sgRNA virus-containing supernatant with 4 μg/ml polybrene. Cells were selected with 10 μg/ml blasticidin after 48 h after infection. Single clones with DOX-inducible Cas9 expression and GPX4 knockout were amplified and used.

### Generation of Constitutive CRISPR/Cas9 Mediated Knockout

Keap1, NRF2 and SREBP1 depleted cells were generated with a CRISPR/Cas9-mediated knockout system. sgRNA sequences were cloned into LentiCRISPRV2. SCD1 depleted cells were generated with CRISPR/Cas9 mediated knockout system, using stable Cas9 expression cells and sanger CRISPR clone. Lentivirus was produced by co-transfection of the lentiviral vector with psPAX2 (Addgene) and VSV-G (Addgene) into 293T cells using PEI. Infected cells were selected in puromycin-containing medium before proceeding to experiments. sgRNA sequences used in this study are listed below:

TABLE 2
sgRNA Sequences

| Gene | sgRNA Sequence |
|---|---|
| KEAP1 | AGCCGCCCGCGGTGTAGATC SEQ ID NO. 13 |
| NFE2L2 | GCGACGGAAAGAGTATGAGC SEQ ID NO. 14 |
| SREBF1 | #1: GAGACCTGCCGCCTTCACAG SEQ ID NO. 15 |
| | #2: GGATCTGGTGGTGGGCACTG SEQ ID NO. 16 |
| SCD | #1: GAGACGATGCCCCTCTACTTGG SEQ ID NO. 17 |
| | #2: TACTATTTTGTCAGTGCCCTGG SEQ ID NO. 18 |

### In Vivo Xenograft Mouse Model

17-β-estradiol 60-day release pellets were implanted subcutaneously into the left flank 7 days before tumor inoculation. GPX4 iKO BT474 cells were inoculated by injecting 5×106 cells subcutaneously in the right flank of 6 to 8 weeks old female athymic nu/nu mice. Tumor growth was monitored regularly via external caliper measurements. When tumors reached intended size, mice were divided randomly into 4 groups: (1) Vehicle group (daily i.p. Vehicle and normal diet), (2) CCI-779 group (daily i.p. 2 mg/kg of CCI-779 and normal diet), (3) Dox group (daily i.p. Vehicle and Dox diet), (4) Dox+CCI-779 group (daily i.p. 2 mg/kg of CCI-779 and DOX diet). Mice were given intraperitoneal injections of 0.9% sterile saline or Dox (daily 100 mg/kg body weight, i.p.) for two days, right before CCI-779 treatment. Subsequently, mice were provided with daily Dox diet for Dox group and Dox+CCI-779 group, with or without CCI-779 treatment, as indicated. CCI-779 were dissolved in ethanol and diluted with a solution of 5% Tween 80 and 5% PEG400 in sterile water and administered by i.p. injection. The maximum width (X) and length (Y) of the tumor were measured every day and the volume (V) was calculated using the formula: V=(X2Y)/2. Tumor growth was monitored over time. For all experiments, mice were sacrificed at a pre-determined endpoint. If any tumor exceeded a volume of 2000 mm3, 1.5 cm in diameter, or 10% of body weight, the mice would immediately be euthanized. At the end of the study, mice were euthanized with C02 and tumors were taken for measurement of weight, followed immunohistochemical staining. Results are presented as mean tumor volume ±SD.

For PC-3 tumor models, male athymic nu/nu mice aged 5 to 6 weeks were injected in the right flank with 5×106 PC-3 cells. Tumors were measured with callipers daily. When tumours reached a mean volume of 200 mm3, mice were randomized into 4 groups: (1) Vehicle group (daily i.p. 65% D5W (5% dextrose in water), 5% Tween-80, 30% PEG-400); (2) IKE group (daily i.p. 50 mg/kg IKE dissolved in 65% D5W (5% dextrose in water), 5% Tween-80, 30% PEG-400); (3) CCI-779 group (daily i.p. 2 mg/kg of CCI-779 dissolved in ethanol and diluted with a solution of 5% Tween 80 and 5% PEG400 in sterile water); (4) IKE+CCI-779 group (daily i.p. 50 mg/kg IKE and 2 mg/kg of CCI-779). At the end of the study, mice were euthanized with C02 and tumours were taken for measurement of weight.

### Immunohistochemistry

Formalin-fixed, paraffin-embedded specimens were collected, and a routine H&E slide was first evaluated. Antigen retrieval was performed using a commercially available antigen retrieval system. Immunohistochemical staining was performed on 5 μm-thick paraffin-embedded sections using rabbit anti-GPX4, mouse anti-Ki-67, rabbit anti-PTGS2, and rabbit-anti pS235/236 S6 antibodies with a standard avidin-biotin HRP detection system. Tissues were counterstained with haematoxylin, dehydrated, and mounted.

### Statistical Analyses

All data, if applicable, were expressed as mean+/−SD from at least three independent experiments. Group differences were performed using two-tailed t-test or two-way ANOVA. P<0.05 was considered statistically significant.

### REFERENCES

1. Stockwell B R, et al. (2017) Ferroptosis: A Regulated Cell Death Nexus Linking Metabolism, Redox Biology, and Disease. Cell 171(2):273-285.
2. Dixon S J, et al. (2012) Ferroptosis: an iron-dependent form of nonapoptotic cell death. Cell 149(5):1060-1072.
3. Yang W S, et al. (2014) Regulation of ferroptotic cancer cell death by GPX4. Cell 156(1-2):317-331.
4. Bersuker K, et al. (2019) The CoQ oxidoreductase FSP1 acts parallel to GPX4 to inhibit ferroptosis. Nature 575(7784):688-692.
5. Doll S, et al. (2019) FSP1 is a glutathione-independent ferroptosis suppressor. Nature 575(7784):693-698.
6. Gao M & Jiang X (2018) To eat or not to eat-the metabolic flavor of ferroptosis. Current opinion in cell biology 51:58-64.
7. Friedmann Angeli J P, Krysko D V, & Conrad M (2019) Ferroptosis at the crossroads of cancer-acquired drug resistance and immune evasion. Nature reviews. Cancer 19(7):405-414.
8. Hassannia B, Vandenabeele P, & Vanden Berghe T (2019) Targeting Ferroptosis to Iron Out Cancer. Cancer cell 35(6):830-849.
9. Zhang Y, et al. (2018) BAP1 links metabolic regulation of ferroptosis to tumour suppression. Nature cell biology 20(10):1181-1192.
10. Jiang L, et al. (2015) Ferroptosis as a p53-mediated activity during tumour suppression. Nature 520(7545):57-62.
11. Gao M, et al. (2019) Role of Mitochondria in Ferroptosis. Molecular cell 73(2):354-363 e353.
12. Jennis M, et al. (2016) An African-specific polymorphism in the TP53 gene impairs p53 tumor suppressor function in a mouse model. Genes & development 30(8):918-930.
13. Wang W, et al. (2019) CD8(+) T cells regulate tumour ferroptosis during cancer immunotherapy. Nature 569(7755):270-274.
14. Lang X, et al. (2019) Radiotherapy and Immunotherapy Promote Tumoral Lipid Oxidation and Ferroptosis via Synergistic Repression of SLC7A11. Cancer discovery 9(12):1673-1685.
15. Ye L F, et al. (2020) Radiation-Induced Lipid Peroxidation Triggers Ferroptosis and Synergizes with Ferroptosis Inducers. ACS chemical biology 15(2):469-484.
16. Lei G, et al. (2020) The role of ferroptosis in ionizing radiation-induced cell death and tumor suppression. Cell research 30(2):146-162.
17. Viswanathan V S, et al. (2017) Dependency of a therapy-resistant state of cancer cells on a lipid peroxidase pathway. Nature 547(7664):453-457.
18. Wu J, et al. (2019) Intercellular interaction dictates cancer cell ferroptosis via NF2-YAP signalling. Nature 572(7769):402-406.
19. Hangauer M J, et al. (2017) Drug-tolerant persister cancer cells are vulnerable to GPX4 inhibition. Nature 551(7679):247-250.
20. Shimano H & Sato R (2017) SREBP-regulated lipid metabolism: convergent physiology—divergent pathophysiology. Nature reviews. Endocrinology 13(12):710-730.
21. Horton J D, Goldstein J L, & Brown M S (2002) SREBPs: activators of the complete program of cholesterol and fatty acid synthesis in the liver. The Journal of clinical investigation 109(9):1125-1131.
22. Duvel K, et al. (2010) Activation of a metabolic gene regulatory network downstream of mTOR complex 1. Molecular cell 39(2):171-183.
23. Ruiz R, et al. (2014) Sterol regulatory element-binding protein-1 (SREBP-1) is required to regulate glycogen synthesis and gluconeogenic gene expression in mouse liver. The Journal of biological chemistry 289(9):5510-5517.
24. Ricoult S J, Yecies J L, Ben-Sahra I, & Manning B D (2016) Oncogenic PI3K and K-Ras stimulate de novo lipid synthesis through mTORC1 and SREBP. Oncogene 35(10):1250-1260.
25. Yecies J L, et al. (2011) Akt stimulates hepatic SREBP1c and lipogenesis through parallel mTORC1-dependent and independent pathways. Cell metabolism 14(1):21-32.
26. Zhang Y, et al. (2017) A Pan-Cancer Proteogenomic Atlas of PI3K/AKT/mTOR Pathway Alterations. Cancer cell 31(6):820-832 e823.
27. Fruman D A, et al. (2017) The PI3K Pathway in Human Disease. Cell 170(4):605-635.
28. Saxton R A & Sabatini D M (2017) mTOR Signaling in Growth, Metabolism, and Disease. Cell 169(2):361-371.
29. Baba Y, et al. (2018) Protective effects of the mechanistic target of rapamycin against excess iron and ferroptosis in cardiomyocytes. American journal of physiology. Heart and circulatory physiology 314(3):H659-H668.
30. Rojo de la Vega M, Chapman E, & Zhang D D (2018) NRF2 and the Hallmarks of Cancer. Cancer cell 34(1):21-43.
31. Ichimura Y, et al. (2013) Phosphorylation of p62 activates the Keap1-Nrf2 pathway during selective autophagy. Molecular cell 51(5):618-631.
32. Sun X, et al. (2016) Activation of the p62-Keap1-NRF2 pathway protects against ferroptosis in hepatocellular carcinoma cells. Hepatology 63(1):173-184.
33. Woo Y, Lee H J, Jung Y M, & Jung Y J (2019) mTOR-Mediated Antioxidant Activation in Solid Tumor Radioresistance. Journal of oncology 2019:5956867.
34. Owen J L, et al. (2012) Insulin stimulation of SREBP-1c processing in transgenic rat hepatocytes requires p70 S6-kinase. Proceedings of the National Academy of Sciences of the United States of America 109(40):16184-16189.
35. Porstmann T, et al. (2008) SREBP activity is regulated by mTORC1 and contributes to Akt-dependent cell growth. Cell metabolism 8(3):224-236.
36. Kamisuki S, et al. (2009) A small molecule that blocks fat synthesis by inhibiting the activation of SREBP. Chemistry & biology 16(8):882-892.
37. Tesfay L, et al. (2019) Stearoyl-CoA Desaturase 1 Protects Ovarian Cancer Cells from Ferroptotic Cell Death. Cancer research 79(20):5355-5366.
38. Magtanong L, et al. (2019) Exogenous Monounsaturated Fatty Acids Promote a Ferroptosis-Resistant Cell State. Cell chemical biology 26(3):420-432 e429.
39. Zhang Y, et al. (2019) Imidazole Ketone Erastin Induces Ferroptosis and Slows Tumor Growth in a Mouse Lymphoma Model. Cell chemical biology 26(5):623-633 e629.
40. Jamaspishvili T, et al. (2018) Clinical implications of PTEN loss in prostate cancer. Nature reviews. Urology 15(4):222-234.
41. Gao M, Monian P, Quadri N, Ramasamy R, & Jiang X (2015) Glutaminolysis and Transferrin Regulate Ferroptosis. Molecular cell 59(2):298-308.
42. Pavlova N N & Thompson C B (2016) The Emerging Hallmarks of Cancer Metabolism. Cell metabolism 23(1):27-47.
43. Hoxhaj G & Manning B D (2019) The PI3K-AKT network at the interface of oncogenic signalling and cancer metabolism. Nature reviews. Cancer.
44. Zuber J, et al. (2011) Toolkit for evaluating genes required for proliferation and survival using tetracycline-regulated RNAi. Nature biotechnology 29(1):79-83.

We claim:

1. A method of treating a tumor in a mammalian subject in need thereof, the method comprising administering to the subject an effective amount of both (a) an inhibitor of the PI3K/Akt/MTOR signaling axis and (b) an inducer of ferroptosis, thereby reducing the growth of and/or causing regression of the tumor in the subject.

2. The method of claim 1, wherein the subject has a tumor with an activating mutation in the PI3K-PTEN-AKT-mTOR pathway or a tumor with a PTEN deletion.

3. The method of claim 1, wherein the subject has a tumor with an activating mutation in the PI3K-PTEN-AKT-mTOR pathway.

4. The method of claim 1, wherein the subject has a tumor with a PTEN deletion.

5. The method of any of the preceding claims, wherein the subject has a breast tumor.

6. The method of claim 5, wherein the subject has a breast tumor with an activating mutation in the PI3K-PTEN-AKT-mTOR pathway.

7. The method of any of claims 1 to 4, wherein the subject has a prostate tumor.

8. The method of claim 7, wherein the subject has a prostate tumor with a PTEN deletion.

9. The method of any of the preceding claims, wherein the inhibitor of the PI3K/Akt/MTOR signaling axis is selected from the group consisting of a PI3K inhibitor, an Akt inhibitor, an MTOR inhibitor, an MTORC1 inhibitor, an SREBP inhibitor and SCD1 inhibitor.

10. The method of any of the preceding claims, wherein the inhibitor of the PI3K/Akt/MTOR signaling axis is a PI3K inhibitor.

11. The method of claim 10, where in the PI3K inhibitor is selected from the group consisting of GDC-0941, SAR245409, SAR245408, BYL-719, GDC-0980, wortmannin, Ly294002, demethoxyviridin, perifosine, delalisib, idelaisib, PX-866, IPI-145, BAY 80-6946, BEZ235, RP6530, TGR 1202, RP5264, SF1126, INK1117, BKM120, Palomid 529, GSK1059615, ZSTK474, PWT33597, IC87114, TG100-115, CAL263, RP6503, PI-103, GNE-477, CUDC-907 and AEZS-136.

12. The method of any of claims 1 to 9, wherein the inhibitor of the PI3K/Akt/MTOR signaling axis is an Akt inhibitor.

13. The method of claim 12, where in the Akt inhibitor is selected from the group consisting of MK-2206, MK-2206, perifosine, GSK690693, ipatasertib (GDC-0068), AZD5365, afuresertib (GSK2110183), At13148, PF-04691502, AT7867, triciribine, CCT128930, A-674563, PHT0427, miltefosine, honokiol, and TIC10.

14. The method of any of claims 1 to 9, wherein the inhibitor of the PI3K/Akt/MTOR signaling axis is an MTOR inhibitor.

15. The method of claim 14, wherein the MTOR inhibitor is selected from the group consisting of SAR245409, GDC-0980, CCI-779, KU-0063794, rapamycin, epigallocatechin gallate (EGCG), caffeine, curcumin, resveratrol, sirolimus, temsirolimus, everolimus, and ridaforolimus.

16. The method of any of claims 1 to 9, wherein the inhibitor of the PI3K/Akt/MTOR signaling axis is an MTORC1 inhibitor.

17. The method of claim 16, where in the MTORC1 inhibitor is selected from the group consisting of Temsirolimus (CCI-779), Torin and a short hairpin RNA (shRNA) inhibitor of RAPTOR.

18. The method of any of claims 1 to 9, wherein the inhibitor of the PI3K/Akt/MTORC1 signaling axis is a an SREBP inhibitor.

19. The method of claim 18, where in the SREBP inhibitor is Fatostatin A.

20. The method of any of claims 1 to 9, wherein the inhibitor of the PI3K/Akt/MTORC1 signaling axis is an SCD1 inhibitor.

21. The method of claim 20, where in the SCD1 inhibitor is CAY10566.

22. The method of any of the preceding claims, wherein the inducer of ferroptosis is selected from the group consisting of RSL3, erastin, imidazole ketone erastin (IKE), sulfasalazine, sorafenib, altretamine, artesunate, ML-162 and ML-210.

23. A therapeutic composition comprising: (a) an inhibitor of the PI3K/Akt/MTOR signaling axis, (b) an inducer of ferroptosis and (c) a therapeutically acceptable carrier, for use in treatment of a tumor in a subject in need thereof.

24. The combination of (a) an inhibitor of the PI3K/Akt/MTOR signaling axis and (b) an inducer of ferroptosis, for use in treatment of a tumor in a subject in need thereof.

25. The composition of claim 23 or the combination of claim 24, wherein the subject has a tumor with an activating mutation in the PI3K-PTEN-AKT-mTOR pathway or a tumor with a PTEN deletion.

26. The composition of claim 23 or the combination of claim 24, wherein the subject has a tumor with an activating mutation in the PI3K-PTEN-AKT-mTOR pathway.

27. The composition of claim 23 or the combination of claim 24, wherein the subject has a tumor with a PTEN deletion.

28. The composition of claim 23 or the combination of claim 24, wherein the subject has a breast tumor.

29. The composition of claim 23 or the combination of claim 24, wherein the subject has a breast tumor with an activating mutation in the PI3K-PTEN-AKT-mTOR pathway.

30. The composition of claim 23 or the combination of claim 24, wherein the subject has a prostate tumor.

31. The composition of claim 23 or the combination of claim 24, wherein the subject has a prostate tumor with a PTEN deletion.

32. The composition or combination of any of claims 23-31, wherein the inhibitor of the PI3K/Akt/MTOR signaling axis is selected from the group consisting of a PI3K inhibitor, an Akt inhibitor, an MTOR inhibitor, an MTORC1 inhibitor, an SREBP inhibitor and SCD1 inhibitor.

33. The composition or combination of any of claims 23-31, wherein the inhibitor of the PI3K/Akt/MTOR signaling axis is a PI3K inhibitor.

34. The composition or combination of claim 33, where in the PI3K inhibitor is selected from the group consisting of GDC-0941, SAR245409, SAR245408, BYL-719, GDC-0980, wortmannin, Ly294002, demethoxyviridin, perifosine, delalisib, idelaisib, PX-866, IPI-145, BAY 80-6946, BEZ235, RP6530, TGR 1202, RP5264, SF1126, INK1117, BKM120, Palomid 529, GSK1059615, ZSTK474, PWT33597, IC87114, TG100-115, CAL263, RP6503, PI-103, GNE-477, CUDC-907 and AEZS-136.

35. The composition or combination of any of claims 23-31, wherein the inhibitor of the PI3K/Akt/MTOR signaling axis is an Akt inhibitor.

36. The composition or combination of any of claim 35, wherein in the Akt inhibitor is selected from the group consisting of MK-2206, MK-2206, perifosine, GSK690693, ipatasertib (GDC-0068), AZD5365, afuresertib (GSK2110183), At13148, PF-04691502, AT7867, triciribine, CCT128930, A-674563, PHT0427, miltefosine, honokiol, and TIC10.

37. The composition or combination of any of claims 23-31, wherein the inhibitor of the PI3K/Akt/MTOR signaling axis is an MTOR inhibitor.

38. The composition or combination of claim 37, wherein the MTOR inhibitor is selected from the group consisting of SAR245409, GDC-0980, CCI-779, KU-0063794, rapamycin, epigallocatechin gallate (EGCG), caffeine, curcumin, resveratrol, sirolimus, temsirolimus, everolimus, and ridaforolimus.

39. The composition or combination of any of claims 23-31, wherein the inhibitor of the PI3K/Akt/MTOR signaling axis is an MTORC1 inhibitor.

40. The composition or combination of claim 39, where in the MTORC1 inhibitor is selected from the group consisting of Temsirolimus (CCI-779), Torin and a short hairpin RNA (shRNA) inhibitor of RAPTOR.

41. The composition or combination of any of claims 23-31, wherein the inhibitor of the PI3K/Akt/MTORC1 signaling axis is a an SREBP inhibitor.

42. The composition or combination of claim 41, where in the SREBP inhibitor is Fatostatin A.

43. The composition or combination of any of claims 23-31, wherein the inhibitor of the PI3K/Akt/MTORC1 signaling axis is an SCD1 inhibitor.

44. The composition or combination of any of claim 43, where in the SCD1 inhibitor is CAY10566.

45. The composition or combination of any of claims 23-44, wherein the inducer of ferroptosis is selected from the group consisting of RSL3, erastin, imidazole ketone erastin (IKE), sulfasalazine, sorafenib, altretamine, artesunate, ML-162 and ML-210.


SEQUENCE LISTING (19 sequences)

SEQ ID NO: 1 - DNA, 20 bp, Artificial Sequence
  Description of Artificial Sequence Synthetic primer
ctcttccagccttccttcct

SEQ ID NO: 2 - DNA, 20 bp, Artificial Sequence
  Description of Artificial Sequence Synthetic primer
agcactgtgttggcgtacag

SEQ ID NO: 3 - DNA, 19 bp, Artificial Sequence
  Description of Artificial Sequence Synthetic primer
gctgctgaccgacatcgaa

SEQ ID NO: 4 - DNA, 20 bp, Artificial Sequence
  Description of Artificial Sequence Synthetic primer
gggtgggtcaaataggccag

SEQ ID NO: 5 - DNA, 23 bp, Artificial Sequence
  Description of Artificial Sequence Synthetic primer
aactccaaggacacagtcaccat

SEQ ID NO: 6 - DNA, 20 bp, Artificial Sequence
  Description of Artificial Sequence Synthetic primer
cagctgctccacgaactcaa

SEQ ID NO: 7 - DNA, 22 bp, Artificial Sequence
  Description of Artificial Sequence Synthetic primer
ggatgggcggaatggtctcttt

SEQ ID NO: 8 - DNA, 23 bp, Artificial Sequence
  Description of Artificial Sequence Synthetic primer
gccagcctgtcgtcctcaatgtc

SEQ ID NO: 9 - DNA, 20 bp, Artificial Sequence
  Description of Artificial Sequence Synthetic primer
aacgccagcgggagcacatc

SEQ ID NO: 10 - DNA, 20 bp, Artificial Sequence
  Description of Artificial Sequence Synthetic primer
ttgcaggcgccacctcatcg

SEQ ID NO: 11 - DNA, 20 bp, Artificial Sequence
  Description of Artificial Sequence Synthetic primer
ccggacacggtcacccgttg

SEQ ID NO: 12 - DNA, 20 bp, Artificial Sequence
  Description of Artificial Sequence Synthetic primer
cgccttgcacgctagctggt

SEQ ID NO: 13 - DNA, 20 bp, Artificial Sequence
  Description of Artificial Sequence Synthetic oligonucleotide
agccgcccgcggtgtagatc

SEQ ID NO: 14 - DNA, 20 bp, Artificial Sequence
  Description of Artificial Sequence Synthetic oligonucleotide
gcgacggaaagagtatgagc

SEQ ID NO: 15 - DNA, 20 bp, Artificial Sequence
  Description of Artificial Sequence Synthetic oligonucleotide
gagacctgccgccttcacag

SEQ ID NO: 16 - DNA, 20 bp, Artificial Sequence
  Description of Artificial Sequence Synthetic oligonucleotide
ggatctggtggtgggcactg

SEQ ID NO: 17 - DNA, 22 bp, Artificial Sequence
  Description of Artificial Sequence Synthetic oligonucleotide
gagacgatgcccctctacttgg

SEQ ID NO: 18 - DNA, 22 bp, Artificial Sequence
  Description of Artificial Sequence Synthetic oligonucleotide
tactattttgtcagtgccctgg

SEQ ID NO: 19 - DNA, 20 bp, Artificial Sequence
  Description of Artificial Sequence Synthetic oligonucleotide
cacgcccgatacgctgagtg